(19) United States
(12) Patent Application Publication
(10) Pub. No.: US 2026/0041632 A1
(43) Pub. Date: Feb. 12, 2026

(54) A PHARMACEUTICAL COMPOSITION OF SALBUTAMOL AND PHARMACEUTICAL GREEN PROPELLANT

(71) Applicant: Cipla Limited, Mumbai (IN)

(72) Inventors: Kiran ROTE, Mumbai (IN); Pradeep BHADAURIA, Weston, FL (US)

(21) Appl. No.: 19/102,198

(22) PCT Filed: Aug. 9, 2023
(86) PCT No.: PCT/IN2023/050762

§ 371 Date: Feb. 7, 2025

(30) Foreign Application Priority Data
Aug. 10, 2022 (IN) 202221045748
Aug. 10, 2022 (IN) 202221045818

(51) Int. Cl. A61K 9/00; A61K 9/08; A61K 9/10; A61K 31/137; A61K 47/10; A61K 47/12

(52) U.S. Cl. CPC A61K 9/008; A61K 9/0078; A61K 9/08; A61K 9/10; A61K 31/137; A61K 47/10; A61K 47/12

(57) ABSTRACT

Disclosed is a pharmaceutical composition for inhalation comprising salbutamol or enantiomer or salt or ester thereof and a pharmaceutically acceptable green propellant. Particularly, disclosed is a pharmaceutical composition for inhalation comprising salbutamol or enantiomer or salt or ester thereof and a pharmaceutically acceptable green propellant HFA-152a and/or HFO-1234ze(E). Also disclosed processes of preparing such compositions.

### PRIORITY DETAILS

This application claims the benefit of Indian Provisional Application No. 202221045748 and Indian Provisional Application No. 202221045818 filed on Aug. 10, 2022, which is hereby incorporated by reference in its entirety.

### TECHNICAL FIELD OF INVENTION

The invention relates to a pharmaceutical composition for inhalation comprising salbutamol or enantiomer or salt or ester thereof and a pharmaceutically acceptable green propellant. Particularly, disclosed is a pharmaceutical composition for inhalation comprising salbutamol or enantiomer or salt or ester thereof and a pharmaceutically acceptable green propellant HFA-152a and/or HFO-1234ze(E). Also disclosed are processes of preparing such compositions and method of preventing or treating respiratory disorders using such compositions.

### BACKGROUND OF INVENTION

Hydrofluoroalkane (HFAs), have been used as propellants in pressurized metered dose inhalers (pMIs) for many years. They replaced chlorofluorocarbons (CFCs), which were ozone depletion agents. HFAs were introduced in 1987 as an alternative propellant in inhalers. Two HFAs in particular—1,1,1,2-tetrafluoroethane (HFA-134a) and 1,1,1,2,3,3,3-heptafluoropropane (HFA-227ea) are commonly used as propellants to deliver and aerosolize medicines from pMDIs. These offer many of the same benefits as CFCs for many inhalation device applications—high propellant performance and non-toxicity in humans—but also have the advantage of not interacting with the ozone layer. As a result, HFA-containing drug devices have become widespread across the globe. However, they do have a significant drawback. They have a relatively high global warming potential (GWP), meaning that they trap relatively high levels of solar heat in the atmosphere—in doing so, they contribute significantly to climate change (UK Progress on reducing F-gas Emissions Fifth report of session 2017-19 published on 25 Apr. 2018 by authority of the House of Commons). GWP is the heat absorbed by any greenhouse gas in the atmosphere, as a multiple of the heat that would be absorbed by the same mass of carbon dioxide (CO₂). The GWP of CO₂ is 1. For other gases, it depends on the gas and the time frame. The higher the figure, the higher the GWP. 1,1,1,2-Tetrafluoroethane (HFC-134a), which is commonly used in pMDIs, has a GWP of 1430, meaning it absorbs 1430 times more heat as the same mass of CO₂.

The inhaler market is expected to grow at 7.1% CAGR to reach more than 2Bn USD mark and hence the number of inhalers to be dispensed accordingly. Therefore, failure to find more sustainable alternative propellants now could mean that patients lose access to vital and effective inhalation drug products in the future. There is growing concern among consumers and among businesses about reducing their own environmental impact. Increasingly, consumers and businesses are taking measures to reduce their environmental footprint. More and more, businesses want to engage and partner with suppliers that are equally “green”.

The pharmaceutical industry has been exploring alternatives to standard propellants for use in inhalation drug delivery devices and other medical devices. The two potential “greener” candidates currently being considered are: 1,1-Difluoroethane (HFA-152a) and 1,3,3,3-Tetrafluoropropene (HFO-1234ze(E)). These next-generation propellants have several key benefits for inhalation drug developers. They are more environmentally friendly than traditional HFAs, exhibiting a lower GWP. HFA-152a, for example has a GWP of 124 compared with the 1430 for traditional inhaler propellant 1,1,1,2-Tetrafluoroethane (HFC-134a). HFO-1234ze(E), meanwhile has a GWP of less than 1, lower even than CO₂. These GWP figures mean that, when in the atmosphere, both HFA-152a and HFO-1234ze(E) do not trap as much heat from the sun, resulting in a less significant global heating effect. In addition, they have a far shorter atmospheric life (AL) compared with existing propellants. HFA-152a, for example, has an AL of 1.4 years, compared with 14 years for currently used inhaler propellants. This means that these new-generation propellants remain in the atmosphere for less time than traditional alternatives, further minimizing their warming effect and environmental impact (https://www.recipharm.com/system/files?file=MDIs%20Tackling%20a%20changing%20climate.pdf).

While they offer important and exciting environmental benefits, the new generation of propellants does have certain disadvantages that need to be considered before they are adopted by the pharmaceutical industry. These include: (1) the use of these new propellants is still in its infancy not just in the pharma space, but elsewhere in the economy, and it is too early to say whether they are straightforward and effective replacements for traditional propellants, (2) as with other gases, both HFA-152a and HFO-1234ze(E) have their own unique properties that means further work have to be done adjusting pMDIs device designs in order to create an effective finished product with an adequate shelf life. Further, while considering new propellant in drug formulation, there are several other factors to be determined such toxicology of propellant, interaction of new propellant with drug and device, interaction of new propellant with manufacturing equipment etc. Even after doing all these, the pharmaceutical companies must pass the regulatory barriers and make the product commercially available to patients at an affordable price.

Salbutamol or albuterol is approved as pMDI in the USA under the brand name VENTOLIN HFA, PROAIR HFA and PROVENTIL HFA. The active component of Ventolin HFA is albuterol sulfate, USP, the racemic form of albuterol and is supplied as VENTOLIN HFA is a blue plastic inhaler with a blue cap containing a pressurized metered-dose aerosol canister fitted with a counter. Each canister contains a microcrystalline suspension of albuterol sulfate in propellant HFA-134a (1,1,1,2-tetrafluoroethane). It contains no other excipients. PROVENTIL HFA Inhalation Aerosol is a pressurized metered-dose aerosol unit for oral inhalation. It contains a microcrystalline suspension of albuterol sulfate in propellant HFA-134a (1,1,1,2-tetrafluoroethane), ethanol, and oleic acid. PROAIR HFA Inhalation Aerosol is a pressurized metered-dose aerosol unit with a dose counter. PROAIR HFA is for oral inhalation only. It contains a microcrystalline suspension of albuterol sulfate in propellant HFA-134a (1, 1, 1, 2-tetrafluoroethane) and ethanol. Levosalbutamol or Levalbuterol (the (R)-enantiomer of the drug substance racemic albuterol) is approved as pMDI in USA under the brand name XOPENEX HFA which is a suspension of micronized levalbuterol tartrate, propellant HFA-134a (1,1,1,2-tetrafluoroethane), Dehydrated Alcohol USP, and Oleic Acid NF. After priming with 4 actuations, each actuation of the inhaler delivers 67.8 mcg of levalbuterol tartrate (equivalent to 51.6 mcg of levalbuterol free base) from the valve and 59 mcg of levalbuterol tartrate (equivalent to 45 mcg of levalbuterol free base) from the actuator mouthpiece. Each 15 g canister provides 200 actuations (or inhalations). Levosalbutamol is also approved as an inhalation Solution is supplied in unit-dose vials and requires no dilution before administration by nebulization. Each 3 mL unit-dose vial contains 0.63 mg of levalbuterol (as 0.73 mg of levalbuterol HCl) or 1.25 mg of levalbuterol (as 1.44 mg of levalbuterol HCl), sodium chloride to adjust tonicity, and sulfuric acid to adjust the pH to 4.0 (3.3 to 4.5).

The U.S. Pat. Nos. 9,114,164; 9,517,216; 10,959,965; US patent application 2021/0244688A1 and US patent application 2021/0244688A1 disclosed aerosol composition of salbutamol sulphate with HFA-152a.

However, there is a need for a pMDI aerosol formulation that has a reduced GWP in comparison with HFA-134a and HFA-227ea, that has acceptable flammability and toxicity performance, which forms stable suspensions or solutions with salbutamol or levosalbutamol or salt or ester thereof.

### SUMMARY OF INVENTION

In the first aspect of the invention, disclosed is a pharmaceutical composition for inhalation comprising salbutamol or enantiomer or salt or ester thereof and a pharmaceutically acceptable green propellant.

In another aspect, disclosed is a pharmaceutical composition for inhalation comprising salbutamol or enantiomer or salt or ester thereof and a pharmaceutically acceptable green propellant HFA-152a. In one embodiment, the HFA-152a is present in a concentration of at least 30% or 40% or 50% or 60% or 70% or 80% or 90% or 99% in the composition. In yet another embodiment, the HFA-152a is present in a concentration of at least 90% in the composition. In yet another embodiment, the composition contains less than 1000 ppm of water based on the total weight of the pharmaceutical composition or the composition contains less than 400 ppm of water based on the total weight of the pharmaceutical composition or the composition contains less than 1 ppm of water based on the total weight of the pharmaceutical composition. In yet another embodiment, the composition contains less than 1000 ppm of oxygen based on the total weight of the pharmaceutical composition or wherein the composition contains less than 1 ppm of oxygen based on the total weight of the pharmaceutical composition or the composition contains less than 0.5 ppm of oxygen based on the total weight of the pharmaceutical composition. In still another embodiment, the propellant HFA-152a is more than 90% or 95% or 99% or 99.5% or 99.99% pure. In still another embodiment, the propellant HFA-152a does not have any substantial impurities or free from any impurities or the propellant HFA-152a contains less than 0.5 to 10 ppm of unsaturated impurities or less than 0.25 ppm of unsaturated impurities. The composition further comprises a pharmaceutically acceptable excipient.

In another embodiment, disclosed is a pharmaceutical composition for inhalation comprising salbutamol or enantiomer or salt or ester thereof and a pharmaceutically acceptable green propellant HFO-1234ze(E). The HFO-1234ze(E) is present in a concentration of at least 30% or 40% or 50% or 60% or 70% or 80% or 90% or 99% in the composition. More preferably, the HFO-1234ze(E) is present in a concentration of at least 90% in the composition. In another embodiment, the composition contains less than 1000 ppm of water based on the total weight of the pharmaceutical composition or the composition contains less than 400 ppm of water based on the total weight of the pharmaceutical composition, or the composition contains less than 1 ppm of water based on the total weight of the pharmaceutical composition. In another embodiment, the composition contains less than 1000 ppm of oxygen based on the total weight of the pharmaceutical composition, or the composition contains less than 1 ppm of oxygen based on the total weight of the pharmaceutical composition, or the composition contains less than 0.5 ppm of oxygen based on the total weight of the pharmaceutical composition. In another embodiment, the propellant HFO-1234ze(E) is more than 90% or 95% or 99% or 99.5% or 99.99% pure. Preferably, the propellant HFO-1234ze(E) does not have any substantial impurities or free from any impurities. More preferably, the propellant HFO-1234ze(E) contains less than 0.5 to 10 ppm of unsaturated impurities or less than 0.25 ppm of unsaturated impurities, or the propellant HFO-1234ze(E) contains less than 10 ppm of HFO-1234ze(Z), or the propellant HFO-1234ze(E) contains less than 5 ppm of HFO-1234ze(Z). The composition further comprises a pharmaceutically acceptable excipient.

In yet another embodiment, disclosed is a pharmaceutical composition for inhalation comprising salbutamol sulphate and a pharmaceutically acceptable green propellant which is combination of HFO-1234ze(E) and HFA-152a. In another embodiment, disclosed is a pharmaceutical composition for inhalation comprising salbutamol or enantiomer or salt or ester thereof, a pharmaceutically acceptable green propellant HFA-152a and/or HFO-1234ze(E) and a surfactant selected from group consisting of oleic acid, polyvinylpyrrolidone of different K grades, lecithin, tween 20, sorbitan trioleate, sorbitan mono oleate, tween 80, polyethylene glycol ranging from 200-1500 grade and combination thereof. In another embodiment, disclosed is a pharmaceutical composition for inhalation comprising salbutamol or enantiomer or salt or ester thereof, a pharmaceutically acceptable green propellant HFA-152a and/or HFO-1234ze(E) and oleic acid. In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol and oleic acid.

Disclosed herein are also the process of preparing such a composition. In one embodiment, the rate of sedimentation of active agent in propellant medium depends on the process of manufacturing of the composition. In another embodiment, the compositions of this invention are manufactured by (1) sonication process, or (2) single stage filing process, or (3) dual stage filing process.

The compositions of this inventions are suitable for oral or nasal inhalation route. The administration or delivery of compositions can be done using pressurized metered dose inhaler (pMDI) or breath actuated inhaler (BAI) or nebulizer (Neb). The invention compositions can be used for preventing or treating respiratory disorders using such compositions.

### BRIEF DESCRIPTION OF FIGURES

FIG. 1: FIGURE shows visual images of vial having suspension composition prepared according to example 14A, 14B, 14C and 14D.

### DETAILED DESCRIPTION

Unless specifically defined otherwise, the technical terms, as used herein, have their normal meaning as understood in the art. The following terms are specifically defined for the sake of clarity.

The term “active agent” is used herein to include an agent such as drug, compound, composition, or other substance that may be used on, or administered to, a human or animal for any purpose, including therapeutic, pharmaceutical, pharmacological, diagnostic, cosmetic, and prophylactic agents and immunomodulators. Active agents may be used interchangeably with the terms drug, pharmaceutical API, medicament, drug substance, or therapeutic agent.

“Mass median aerodynamic diameter” or “MMAD” as used herein refers to the aerodynamic diameter of an aerosol below which 50% of the mass of the aerosol contains particles with an aerodynamic diameter smaller than the MMAD, with the MMAD being calculated according to monograph 601 of the United States Pharmacopeia (“USP).

“Extra fine particle” as used herein refers to the particle having MMAD less than 2 μm or preferably less than 1 μm.

“Extra fine aerosol formulation or composition or extra fine suspension” as used herein refers to the composition having more than 50% or 60% or 70% or 80% or 90% or 95% of particle having MMAD less than 2 μm or preferably less than 1 μm.

A “therapeutically effective amount” is the amount of compound which achieves a therapeutic effect by inhibiting a disease or disorder in a patient or by prophylactically inhibiting or preventing the onset of a disease or disorder, specifically respiratory disorder. A therapeutically effective amount may be an amount which relieves to some extent one or more symptoms of a disease or disorder in a patient; returns to normal either partially or completely one or more physiological or biochemical parameters associated with or causative of the disease or disorder; and/or reduces the likelihood of the onset of the disease or disorder.

The terms “stable” or “chemical stable” refer to composition wherein the individual degradation products of active agent remain below the limits specified by regulatory requirements during the shelf life of the product for human use (e.g., 1% of total chromatographic peak area per ICH guidance Q3B(R2)) and there is acceptable mass balance (e.g., as defined in ICH guidance Q1E) between active agent assay and total degradation products.

The term “substantial” refers to more than 90% or 95% or 99% or 99.99%. For e.g., the propellant substantially free of any impurities means that the impurities in or associated with propellant are less than 10% or less than 5% or less than 1% or absent in the propellant.

The term “% w/w” refers to weight of particular active or excipient by weight of the composition.

The active agent present in the invention is salbutamol or levosalbutamol or salt or ester thereof and both are short-acting bronchodilator used in the treatment and control of several respiratory-related disorders, but particularly asthma and chronic obstructive pulmonary disease (COPD). The drug is conveniently delivered using pMDI or breath actuated MDI (BAI). In particular, the invention relates to salbutamol sulphate, levosalbutamol hydrochloride or levosalbutamol tartrate or combination thereof. The words “salbutamol” and “albuterol” have been used interchangeably throughout the specification. Similarly, the words “levosalbutamol” and “levalbuterol” have been used interchangeably throughout the specification.

In an embodiment, the invention composition comprises about 0.05 to about 0.8% by weight of salbutamol or salt or ester thereof. In another embodiment, the invention composition comprises about 0.05 to about 0.8% by weight of levosalbutamol or salt or ester thereof. In another embodiment, the invention composition comprises about 0.1% or 0.2% or 0.3% or 0.4% or 0.5% or 0.6% or 0.7% or about 0.8% by weight of salbutamol or salt or ester thereof. In another embodiment, the invention composition comprises about 0.1% or 0.2% or 0.3% or 0.4% or 0.5% or 0.6% or 0.7% or about 0.8% by weight of levosalbutamol or salt or ester thereof. In preferred embodiment, the active agent is salbutamol sulphate. In another preferred embodiment, the active agent is levosalbutamol hydrochloride. In another preferred embodiment, the active agent is levosalbutamol tartrate.

There is a need for a pharmaceutical composition containing salbutamol or levosalbutamol or salt or ester thereof which can be delivered using pMDI or BAI and that uses a propellant having a reduced GWP in comparison with HFA-134a and HIFA-227ea. There is also a need for a salbutamol-containing pharmaceutical composition which exhibits improved stability over shelf life. The inventors of instant invention found a stable pharmaceutical composition for inhalation which can be delivered not only by pDMI but also using BAI or nebulizer device wherein the composition is prepared using propellant with low GWP such as HFA-152a and/or HFO-1234ze(E).

HFA-152a (Zephex® 152a) is a new, sustainable medical propellant that has been under development by Koura for several years for use in pMDI's for treatment of respiratory disorders such as asthma and COPD. Below are some specific physicochemical characteristics of HFA-152a. Apart from HFA-152a, hydrofluoroolefins (HFOs), a family of unique products that offer comparable performance to today's most widely used stationary and mobile refrigerants, blowing agents and aerosol propellants. HIFO-1234ze(E) (Solstice) is trans-1,3,3,3-tetrafluoroprop-1-ene. The suffix (E) indicates that it is an isomer. The other isomer (suffix (Z)) is cis-1,3,3,3-tetrafluoroprop-1-ene. The physical properties of the (E) and (Z) isomers are different: both are ultralow GWP molecules with GWP<1.

In first embodiment of the invention, disclosed is a pharmaceutical composition for inhalation comprising salbutamol or enantiomer or salt or ester thereof and a pharmaceutically acceptable green propellant. The compositions of this inventions are suitable for oral or nasal inhalation route. The administration or delivery of compositions can be done using pMDI or BAI or nebulizer (Neb).

A pressurised metered dose inhaler is an inhaler which delivers an aerosolised dose of a pharmaceutical composition using a pressurised liquefied propellant. A pressurised metered dose inhaler typically comprises a canister (or vial) comprising a liquid pharmaceutical composition, a metering valve, an actuator, and a mouthpiece. For stability improvement of composition, various canister specifications such as 19 ml coated canister, 14 ml plain, 14 ml coated can also be used. Similarly, various capacity valves such as 25 mcl, 50 mcl can also be used to minimise the retention of the active agent within the canister. Further, the actuator can be fitted with currently available or known dose counters. To improve the performance of the product through shelf life. use of various valve having gasket such as EPDM, nitrile, COCE, Buna etc can be used. Similar types of canister or valves may be used in case of breath actuated inhaler. The pressurised metered dose inhalers are well known to those of ordinary skill in the art, and many such devices are commercially available, with representative devices including AeroBid Inhaler System (Forest Pharmaceuticals), Atrovent Inhalation Aerosol (Boehringer Ingelheim), FloventR™ (GlaxoSmithKline), Maxair Inhaler (3M), ProventilR™ Inhaler (Schering) and SereventR™ Inhalation Aerosol (GlaxoSmithKline). Further, various breath actuated inhalers present in market are Synchrobreathe™, Redihaler™, Easibreathe™, K-haler® and other available breath actuated inhalers. Also, the invention composition (solution or suspension) can also be delivered using currently available nebulizers such as Respimat®, Philips I-Neb™ (Philips), the Pari LC Sprint (Pari GmbH), the AERx® Pulmonary Delivery System (Aradigm Corp.) and the Pari LC Plus Reusable Nebulizer (Pari GmbH).

When the composition is delivered using pMDI or BAI, in some embodiments, each actuation delivers 120 mcg salbutamol sulphate from the valve and 108 mcg from a mouthpiece which is equivalent to 90 mcg of salbutamol base. In another preferred embodiment, each actuation delivers 67.8 mcg of levalbuterol tartrate (equivalent to 51.6 mcg of levalbuterol free base) from the valve and 59 mcg of levalbuterol tartrate (equivalent to 45 mcg of levalbuterol free base) from the actuator the mouthpiece.

In further embodiments, the pharmaceutical compositions are prepared in the form of a suspension or a solution. The active agents present in the composition are micronized (MMAD between 3 to 5 μm) or extra-fine (MMAD less than 2 or 1 μm). More specifically, the suspension composition comprises micronized active agent and the solution composition comprises extra-fine active agent(s). In extra fine composition, the particle size that is MMAD of active agent is less than 1 μm or 2 μm. Further, for suspension composition of the inventions, the active agent particles have a particle size distribution with a Dv50 (median particle size by volume) value of from about 0.2 m to about 5 μm. More typically, the particles of active agent have a particle size distribution with a Dv50 value of from about 0.7 μm to about 3.0 μm. For instance, the particles of active agent (salbutamol or enantiomer or salt or ester thereof) may have a particle size distribution with a Dv50 value of from 0.9 μm to 1.7 μm, or alternatively from 1.7 μm to 2.7 μm. Often, the particles of salbutamol or enantiomer or salt or ester thereof have a particle size distribution with a Dv50 value of from about 1.1 μm to about 2.6 μm.

The pharmaceutical composition further comprises pharmaceutically acceptable excipient. The pharmaceutically acceptable excipient is selected from the group consisting of, but not limited to, solvent, surfactant, solubilizer, lubricant buffers, pH adjusting agents or combination thereof. The solvent or co-solvents, carrier solvents and adjuvants altogether termed as excipient which serve to solubilise the surfactant or the drug in the propellant and/or inhibit deposition of drug particles on the surfaces of the metered dose inhaler that are contacted by the pharmaceutical composition as it passes from the container in which it is stored to the nozzle outlet. The solvent can be used in an amount between 0.5 to 25.0% or 1 to 10% or 15% by weight of the composition. In general, solvent can be added to composition in q.s. based upon the concentration of other ingredients and total volume of the composition. The solvents examples are, but not limited to ethanol, water, propyl alcohol, isopropyl alcohol or combination thereof. In one embodiment, ethanol is preferred solvent and present in concentration of about 1% or 5% or 10% or 15% by weight of composition.

In an alternate embodiment, the composition comprises water as preferred solvent. In another alternate embodiment, the composition is devoid of water and made as non-aqueous composition, wherein ethanol is preferred solvent. Preferably, the water content is less than 1000 ppm or 400 ppm or 5 ppm or 1 ppm.

The surfactant or solubilizer aids in solubilization or stabilization of salbutamol or enantiomer or salt or ester thereof. Preferred examples of surfactant are, but not limited to, polyvinylpyrrolidone, polyethylene glycol, oleic acid, lecithin, polysorbates or combination thereof. The concentration of surfactant ranges from 0.001% to 2% by weight of composition. The composition may comprise lubricant which is selected from but not limited to glycerol, simethicone, silicon dioxide and magnesium stearate or combination thereof. The lubricant is present in an amount of between 0.001% to 2% by weight of the composition. The preferred concentration is 0.1% by weight of the composition. The person skilled in the art may use pH adjusting agents such as any strong acid like hydrochloric acid or strong base like sodium hydroxide or combination thereof. Further, the person skilled in the art may also add buffers such as weak acid, weak base, strong acid+weak base or weak acid+strong base. For e.g., citric acid, acetic acid, malic acid, tartaric acid, sodium citrate, sodium phosphate, potassium phosphate, glycine and other conventional buffers known to person skilled in the art.

In one preferred embodiment, the active agent in the pharmaceutical composition is salbutamol base or salbutamol sulphate or levalbuterol base or levalbuterol sulphate or levalbuterol tartrate or levalbuterol hydrochloride. In another embodiment the salbutamol sulphate is present in a concentration ranging from 0.05% w/w to 1% w/w or preferably between 0.1% w/w to 0.5% w/w or more preferably between 0.2% w/w to 0.4% w/w of the composition. In preferred embodiment, the salbutamol sulphate present in about 0.16% w/w or about 0.21% w/w of the composition. Similarly, levosalbutamol base or levalbuterol sulphate or levalbuterol tartrate or levalbuterol hydrochloride is present in a concentration ranging from 0.05% w/w to 1% w/w or preferably between 0.1% w/w to 0.5% w/w or more preferably between 0.2% w/w to 0.4% w/w of the composition. In preferred embodiment, the levosalbutamol or levalbuterol sulphate or levalbuterol tartrate or levalbuterol hydrochloride is present in a concentration of about 0.16% w/w or about 0.21% w/w of the composition. Alternatively, the salbutamol sulphate is present in a concentration ranging from 50 mcg to 400 mcg or preferably between 100 mcg to 200 mcg or more preferably about 100 mcg or 200 mcg in the composition. Alternatively, the salbutamol sulphate is present in a concentration of 50 mcg or 60 mcg or 70 mcg or 80 mcg or 90 mcg or 100 mcg or 120 mcg or 140 mcg or 160 mcg or 180 mcg or 200 mcg. Alternatively, levosalbutamol base or levalbuterol sulphate or levalbuterol tartrate or levalbuterol hydrochloride is present a concentration ranging from 50 mcg to 400 mcg or preferably between 100 mcg to 200 mcg or more preferably about 100 mcg or 200 mcg in the composition. Alternatively, levosalbutamol base or levalbuterol sulphate or levalbuterol tartrate or levalbuterol hydrochloride is present in a concentration of 50 mcg or 60 mcg or 70 mcg or 80 mcg or 90 mcg or 100 mcg or 120 mcg or 140 mcg or 160 mcg or 180 mcg or 200 mcg.

In yet another embodiment, when the composition administered using pMDI or BAI, the salbutamol sulphate delivered from a valve of the metered dose inhaler is present in a concentration ranging from 50 mcg to 400 mcg or preferably between 100 mcg to 200 mcg or more preferably about 120 mcg. Similarly, in another embodiment, levosalbutamol base or levalbuterol sulphate or levalbuterol tartrate or levalbuterol hydrochloride delivered from a valve of the metered dose inhaler is present a concentration ranging from 50 mcg to 400 mcg or preferably between 100 mcg to 200 mcg or more preferably about 67.8 mcg. In another embodiment, the composition is in the form of the suspension and wherein the salbutamol or levosalbutamol base or levalbuterol sulphate or levalbuterol tartrate or levalbuterol hydrochloride thereof are in micronized form. Alternatively, the composition is in the form of the extra-fine composition, wherein the more than 50% or 60% or 70% or 80% or 90% or 95% of particle of salbutamol or enantiomer or salt or ester thereof are having aerodynamic diameter less than 2 μm or less than 1 μm. The composition in the form of the extra-fine suspension, where more than 50% or 60% or 70% or 80% or 90% or 95% of particle of salbutamol or enantiomer or salt or ester thereof are having aerodynamic diameter less than 2 μm. When the composition has extra fine active agent, then the composition is referred as solution. Further, in solution composition, the active agent is dissolved in propellant or other excipients.

The pharmaceutical composition of this invention comprises a pharmaceutically acceptable green propellant selected from group of propellants having global warming potential of less than 250, more preferably less than 200 and still more preferably less than 150 or less than 100 or less than 50 or less than 20 or less than 10 or less than 5. In another embodiment, the pharmaceutically acceptable green propellant is selected from HFA-152a or HFO-1234ze(E) or combination thereof.

In one embodiment, the pharmaceutically acceptable green propellant HFA-152a. The green propellant HFA-152a is present in a concentration of at least 30% or 40% or 50% or 60% or 70% or 80% or 90% or 99% in the composition. In one embodiment, the pharmaceutically acceptable green propellant HFA-152a is present in a concentration of at least 90% in the composition. The green propellant HFA-152a is more than 90% or 95% or 99% or 99.5% or 99.99% pure. In another embodiment, the propellant HFA-152a does not have any substantial impurities or is free from any impurities. Preferably, the propellant HFA-152a contains less than 0.5 to 10 ppm of unsaturated impurities or less than 0.25 ppm of unsaturated impurities.

In another embodiment, the composition contains less than 1000 ppm or less than 400 pm of water based on the total weight of the pharmaceutical composition. Preferably, the composition contains less than 1 ppm of water based on the total weight of the pharmaceutical composition. More preferably, the composition contains less than 0.5 ppm of water based on the total weight of the pharmaceutical composition. In yet another embodiment, the composition contains less than 1000 ppm of oxygen based on the total weight of the pharmaceutical composition. Preferably, the composition contains less than 1 ppm of oxygen based on the total weight of the pharmaceutical composition. More preferably, the composition contains less than 0.5 ppm of oxygen based on the total weight of the pharmaceutical composition.

In yet another embodiment, the composition when administered using the metered dose inhaler or breath actuated metered dose inhaler or nebulizer produces a fine particle fraction of the salbutamol or enantiomer or salt or ester thereof which is at least 40.0% weight of the emitted dose of the salbutamol or enantiomer or salt or ester thereof even after storage of the pharmaceutical composition at 500 C. and 75% relative humidity for 30 or 60 or 90 or 180 days. In still another embodiment, the composition when administered using the metered dose inhaler or breath actuated metered dose inhaler or nebulizer produces a fine particle fraction of the salbutamol or enantiomer or salt or ester thereof which is at least 42% weight of the emitted dose of the salbutamol or enantiomer or salt or ester thereof even after storage of the pharmaceutical composition at 50° C. and 75% relative humidity for 30 or 60 or 90 or 180 days as compared to composition comprising salbutamol or enantiomer or salt or ester thereof and a propellant HFA134a or HFA227ea.

In yet another embodiment, the composition when administered using the metered dose inhaler or breath actuated metered dose inhaler or nebulizer, produces a fine particle mass of salbutamol or enantiomer or salt or ester thereof with less variability having relative standard deviation less than 6% or less than 4% over shelf life. In still another embodiment, the composition when administered using the metered dose inhaler or breath actuated metered dose inhaler or nebulizer, produces a fine particle mass of salbutamol or enantiomer or salt or ester thereof with less variability over shelf life as compared to composition comprising salbutamol or enantiomer or salt or ester thereof and a propellant HFA134a or HFA227ea. The relative standard deviation (variability) in fine particle mass for composition comprising salbutamol or enantiomer or salt or ester thereof and a propellant HFA134a or HFA227ea more than 7% or more than 7.5% over shelf life.

In an embodiment, disclosed is a pharmaceutical composition for inhalation comprising salbutamol or enantiomer or salt or ester thereof and a pharmaceutically acceptable green propellant HFA-152a. In another embodiment, the HFA-152a is present in a concentration of at least 30% or 40% or 50% or 60% or 70% or 80% or 90% or 99% in the composition. In yet another embodiment, the HFA-152a is present in a concentration of at least 90% in the composition. In yet another embodiment, the composition contains less than 1000 ppm or less than 400 ppm of water based on the total weight of the pharmaceutical composition or the composition contains less than 1 ppm of water based on the total weight of the pharmaceutical composition or the composition contains less than 0.5 ppm of water based on the total weight of the pharmaceutical composition. In yet another embodiment, the composition contains less than 1000 ppm of oxygen based on the total weight of the pharmaceutical composition or wherein the composition contains less than 1 ppm of oxygen based on the total weight of the pharmaceutical composition or the composition contains less than 0.5 ppm of oxygen based on the total weight of the pharmaceutical composition. In still another embodiment, the propellant HFA-152a is more than 90% or 95% or 99% or 99.5% or 99.99% pure. In still another embodiment, the propellant HFA-152a does not have any substantial impurities or is free from any impurities or the propellant HFA-152a contains less than 0.5 to 10 ppm of unsaturated impurities or less than 0.25 ppm of unsaturated impurities.

In another embodiment, disclosed is a pharmaceutical composition for inhalation comprising salbutamol or enantiomer or salt or ester thereof and a pharmaceutically acceptable green propellant HFA-152a, wherein the composition when administered using the metered dose inhaler produces a fine particle fraction of the salbutamol or enantiomer or salt or ester thereof which is at least 40.0% weight of the emitted dose of the salbutamol or enantiomer or salt or ester thereof even after storage of the pharmaceutical composition at 500 C. and 75% relative humidity for 30 or 60 or 90 or 180 days.

In another embodiment, disclosed is a pharmaceutical composition for inhalation comprising salbutamol or enantiomer or salt or ester thereof and a pharmaceutically acceptable green propellant HFA-152a, wherein the composition when administered using the metered dose inhaler produces a fine particle fraction of the salbutamol or enantiomer or salt or ester thereof which is at least 42% weight of the emitted dose of the salbutamol or enantiomer or salt or ester thereof even after storage of the pharmaceutical composition at 500 C. and 75% relative humidity for 30 or 60 or 90 or 180 days as compared to composition comprising salbutamol or enantiomer or salt or ester thereof and a propellant HFA134a or HFA227ea.

In another embodiment, disclosed is a pharmaceutical composition for inhalation comprising salbutamol sulphate and a pharmaceutically acceptable green propellant HFA-152a. In another embodiment, disclosed is a pharmaceutical composition for inhalation comprising levosalbutamol tartrate and a pharmaceutically acceptable green propellant HFA-152a. In another embodiment, disclosed is a pharmaceutical composition for inhalation comprising levosalbutamol hydrochloride and a pharmaceutically acceptable green propellant HFA-152a. In another embodiment, disclosed is a pharmaceutical composition for inhalation comprising salbutamol sulphate and a pharmaceutically acceptable green propellant HFA-152a as a sole propellant. In another embodiment, disclosed is a pharmaceutical composition for inhalation comprising levosalbutamol tartrate and a pharmaceutically acceptable green propellant HFA-152a as a sole propellant. In another embodiment, disclosed is a pharmaceutical composition for inhalation comprising levosalbutamol hydrochloride and a pharmaceutically acceptable green propellant HFA-152a as a sole propellant.

In an alternate embodiment, disclosed is a pharmaceutical composition for inhalation comprising salbutamol sulphate and a pharmaceutically acceptable green propellant which is combination of HFA-152a and HFA134a. In another embodiment, disclosed is a pharmaceutical composition for inhalation comprising levosalbutamol tartrate and a pharmaceutically acceptable green propellant which is combination of HFA-152a and HFA134a. In another embodiment, disclosed is a pharmaceutical composition for inhalation comprising levosalbutamol hydrochloride and a pharmaceutically acceptable green propellant which is combination of HFA-152a and HFA134a.

In another embodiment, disclosed is a pharmaceutical composition for inhalation comprising salbutamol sulphate and a pharmaceutically acceptable green propellant which is combination of HFA-152a and HFA227ea. In another embodiment, disclosed is a pharmaceutical composition for inhalation comprising levosalbutamol tartrate and a pharmaceutically acceptable green propellant which is combination of HFA-152a and HFA227ea. In another embodiment, disclosed is a pharmaceutical composition for inhalation comprising levosalbutamol hydrochloride and a pharmaceutically acceptable green propellant which is combination of HFA-152a and HFA227ea.

In another embodiment, disclosed is a pharmaceutical composition for inhalation comprising salbutamol sulphate and a pharmaceutically acceptable green propellant which is combination of HFA-152a and HFA134a, wherein HFA-152a is present in a concentration ranging from 20% to 90% by weight of composition and HFA134a is present in a concentration ranging from 20% to 90% by weight of composition. In another embodiment, disclosed is a pharmaceutical composition for inhalation comprising salbutamol sulphate and a pharmaceutically acceptable green propellant which is combination of HFA-152a and HFA134a, wherein HFA-152a is present in a concentration ranging from 70% by weight of composition and HFA134a is present in a concentration ranging from 30% by weight of composition. In another embodiment, disclosed is a pharmaceutical composition for inhalation comprising salbutamol sulphate and a pharmaceutically acceptable green propellant which is combination of HFA-152a and HFA134a, wherein HFA-152a is present in a concentration ranging from 30% by weight of composition and HFA134a is present in a concentration ranging from 70% by weight of composition. In another embodiment, disclosed is a pharmaceutical composition for inhalation comprising levosalbutamol tartrate and a pharmaceutically acceptable green propellant which is combination of HFA-152a and HFA134a, wherein HFA-152a is present in a concentration ranging from 20% to 90% by weight of composition and HFA134a is present in a concentration ranging from 20% to 90% by weight of composition. In another embodiment, disclosed is a pharmaceutical composition for inhalation comprising levosalbutamol tartrate and a pharmaceutically acceptable green propellant which is combination of HFA-152a and HFA134a, wherein HFA-152a is present in a concentration ranging from 70% by weight of composition and HFA134a is present in a concentration ranging from 30%% by weight of composition. In another embodiment, disclosed is a pharmaceutical composition for inhalation comprising levosalbutamol tartrate and a pharmaceutically acceptable green propellant which is combination of HFA-152a and HFA134a, wherein HFA-152a is present in a concentration ranging from 30%% by weight of composition and HFA134a is present in a concentration ranging from 70% by weight of composition. In another embodiment, disclosed is a pharmaceutical composition for inhalation comprising levosalbutamol hydrochloride and a pharmaceutically acceptable green propellant which is combination of HFA-152a and HFA134a, wherein HFA-152a is present in a concentration ranging from 20% to 90% by weight of composition and HFA134a is present in a concentration ranging from 20% to 90% by weight of composition. In another embodiment, disclosed is a pharmaceutical composition for inhalation comprising levosalbutamol hydrochloride and a pharmaceutically acceptable green propellant which is combination of HFA-152a and HFA134a, wherein HFA-152a is present in a concentration ranging from 70% by weight of composition and HFA134a is present in a concentration ranging from 30% by weight of composition. In another embodiment, disclosed is a pharmaceutical composition for inhalation comprising levosalbutamol hydrochloride and a pharmaceutically acceptable green propellant which is combination of HFA-152a and HFA134a, wherein HFA-152a is present in a concentration ranging from 30% by weight of composition and HFA134a is present in a concentration ranging from 70% by weight of composition.

In another embodiment, disclosed is a pharmaceutical composition for inhalation comprising salbutamol or enantiomer or salt or ester thereof and a pharmaceutically acceptable green propellant HFO-1234ze(E). The HFO-1234ze(E) is present in a concentration of at least 30% or 40% or 50% or 60% or 70% or 80% or 90% or 99% in the composition. More preferably, the HFO-1234ze(E) is present in a concentration of at least 90% in the composition. In another embodiment, the composition contains less than 1000 ppm or 400 pm of water based on the total weight of the pharmaceutical composition or the composition contains less than 1 ppm of water based on the total weight of the pharmaceutical composition, or the composition contains less than 0.5 ppm of water based on the total weight of the pharmaceutical composition. In another embodiment, the composition contains less than 1000 ppm of oxygen based on the total weight of the pharmaceutical composition, or the composition contains less than 1 ppm of oxygen based on the total weight of the pharmaceutical composition, or the composition contains less than 0.5 ppm of oxygen based on the total weight of the pharmaceutical composition. In another embodiment, the propellant HFO-1234ze(E) is more than 90% or 95% or 99% or 99.5% or 99.99% pure. Preferably, the propellant HFO-1234ze(E) does not have any substantial impurities or free from any impurities. More preferably, the propellant HFO-1234ze(E) contains less than 0.5 to 10 ppm of unsaturated impurities or less than 0.25 ppm of unsaturated impurities, or the propellant HFO-1234ze(E) contains less than 10 ppm of HFO-1234ze(Z), or the propellant HFO-1234ze(E) contains less than 5 ppm of HFO-1234ze(Z).

In yet another embodiment, disclosed is a pharmaceutical composition for inhalation comprising salbutamol or enantiomer or salt or ester thereof and a pharmaceutically acceptable green propellant HFO-1234ze(E), wherein the composition when administered using the metered dose inhaler or breath actuated metered dose inhaler or nebulizer produces a fine particle fraction of the salbutamol or enantiomer or salt or ester thereof which is at least 40.0% weight of the emitted dose of the salbutamol or enantiomer or salt or ester thereof even after storage of the pharmaceutical composition at 500 C. and 75% relative humidity for 30 or 60 or 90 or 180 days.

In yet another embodiment, disclosed is a pharmaceutical composition for inhalation comprising salbutamol or enantiomer or salt or ester thereof and a pharmaceutically acceptable green propellant HFO-1234ze(E), wherein the composition when administered using the metered dose inhaler or breath actuated metered dose inhaler or nebulizer produces a fine particle fraction of the salbutamol or enantiomer or salt or ester thereof which is at least 42% weight of the emitted dose of the salbutamol or enantiomer or salt or ester thereof even after storage of the pharmaceutical composition at 50° C. and 75% relative humidity for 30 or 60 or 90 or 180 days as compared to composition comprising salbutamol or enantiomer or salt or ester thereof and a propellant HFA134a or HFA227ea.

In yet another embodiment, the composition when administered using the metered dose inhaler or breath actuated metered dose inhaler or nebulizer, produces a fine particle mass of salbutamol or enantiomer or salt or ester thereof with less variability having relative standard deviation less than 6.5% over shelf life. In still another embodiment, the composition when administered using the metered dose inhaler or breath actuated metered dose inhaler or nebulizer, produces a fine particle mass of salbutamol or enantiomer or salt or ester thereof with less variability having relative standard deviation less than 6.2% over shelf life as compared to composition comprising salbutamol or enantiomer or salt or ester thereof and a propellant HFA134a or HFA227ea where the relative standard deviation more than 7% or more than 7.5%.

In another embodiment, disclosed is a pharmaceutical composition for inhalation comprising salbutamol sulphate and a pharmaceutically acceptable green propellant HFO-1234ze(E) as a sole propellant. In another embodiment, disclosed is a pharmaceutical composition for inhalation comprising levosalbutamol tartrate and a pharmaceutically acceptable green propellant HFO-1234ze(E) as a sole propellant. In another embodiment, disclosed is a pharmaceutical composition for inhalation comprising levosalbutamol hydrochloride and a pharmaceutically acceptable green propellant HFO-1234ze(E) as a sole propellant.

In alternate embodiment, disclosed is a pharmaceutical composition for inhalation comprising salbutamol sulphate and a pharmaceutically acceptable green propellant which is combination of HFO-1234ze(E) and HFA134a. In another embodiment, disclosed is a pharmaceutical composition for inhalation comprising levosalbutamol tartrate and a pharmaceutically acceptable green propellant which is combination of HFO-1234ze(E) and HFA134a. In another embodiment, disclosed is a pharmaceutical composition for inhalation comprising levosalbutamol hydrochloride and a pharmaceutically acceptable green propellant which is combination of HFO-1234ze(E) and HFA134a.

In alternate embodiment, disclosed is a pharmaceutical composition for inhalation comprising salbutamol sulphate and a pharmaceutically acceptable green propellant which is combination of HFO-1234ze(E) and HFA227ea. In another embodiment, disclosed is a pharmaceutical composition for inhalation comprising levosalbutamol tartrate and a pharmaceutically acceptable green propellant which is combination of HFO-1234ze(E) and HFA227ea. In another embodiment, disclosed is a pharmaceutical composition for inhalation comprising levosalbutamol hydrochloride and a pharmaceutically acceptable green propellant which is combination of HFO-1234ze(E) and HFA227ea.

In another embodiment, disclosed is a pharmaceutical composition for inhalation comprising salbutamol sulphate and a pharmaceutically acceptable green propellant which is combination of HFO-1234ze(E) and HFA134a, wherein HFO-1234ze(E) is present in a concentration ranging from 20% to 90% by weight of composition and HFA134a is present in a concentration ranging from 20% to 90% by weight of composition. In another embodiment, disclosed is a pharmaceutical composition for inhalation comprising salbutamol sulphate and a pharmaceutically acceptable green propellant which is combination of HFO-1234ze(E) and HFA134a, wherein HFO-1234ze(E) is present in a concentration ranging from 70% by weight of composition and HFA134a is present in a concentration ranging from 30% by weight of composition. In another embodiment, disclosed is a pharmaceutical composition for inhalation comprising salbutamol sulphate and a pharmaceutically acceptable green propellant which is combination of HFO-1234ze(E) and HFA134a, wherein HFO-1234ze(E) is present in a concentration ranging from 30% by weight of composition and HFA134a is present in a concentration ranging from 70% by weight of composition. In another embodiment, disclosed is a pharmaceutical composition for inhalation comprising levosalbutamol tartrate and a pharmaceutically acceptable green propellant which is combination of HFO-1234ze(E) and HFA134a, wherein HFO-1234ze(E) is present in a concentration ranging from 20% to 90% by weight of composition and HFA134a is present in a concentration ranging from 20% to 90% by weight of composition. In another embodiment, disclosed is a pharmaceutical composition for inhalation comprising levosalbutamol tartrate and a pharmaceutically acceptable green propellant which is combination of HFO-1234ze(E) and HFA134a, wherein HFO-1234ze(E) is present in a concentration ranging from 70% by weight of composition and HFA134a is present in a concentration ranging from 30%% by weight of composition. In another embodiment, disclosed is a pharmaceutical composition for inhalation comprising levosalbutamol tartrate and a pharmaceutically acceptable green propellant which is combination of HFO-1234ze(E) and HFA134a, wherein HFO-1234ze(E) is present in a concentration ranging from 30%% by weight of composition and HFA134a is present in a concentration ranging from 70% by weight of composition. In another embodiment, disclosed is a pharmaceutical composition for inhalation comprising levosalbutamol hydrochloride and a pharmaceutically acceptable green propellant which is combination of HFO-1234ze(E) and HFA134a, wherein HFO-1234ze(E) is present in a concentration ranging from 20% to 90% by weight of composition and HFA134a is present in a concentration ranging from 20% to 90% by weight of composition. In another embodiment, disclosed is a pharmaceutical composition for inhalation comprising levosalbutamol hydrochloride and a pharmaceutically acceptable green propellant which is combination of HFO-1234ze(E) and HFA134a, wherein HFO-1234ze(E) is present in a concentration ranging from 70% by weight of composition and HFA134a is present in a concentration ranging from 30% by weight of composition. In another embodiment, disclosed is a pharmaceutical composition for inhalation comprising levosalbutamol hydrochloride and a pharmaceutically acceptable green propellant which is combination of HFO-1234ze(E) and HFA134a, wherein HFO-1234ze(E) is present in a concentration ranging from 30% by weight of composition and HFA134a is present in a concentration ranging from 70% by weight of composition.

In yet another embodiment, disclosed is a pharmaceutical composition for inhalation comprising salbutamol sulphate and a pharmaceutically acceptable green propellant which is combination of HFO-1234ze(E) and HFA-152a. In another embodiment, disclosed is a pharmaceutical composition for inhalation comprising levosalbutamol tartrate and a pharmaceutically acceptable green propellant which is combination of HFO-1234ze(E) and HFA-152a. In another embodiment, disclosed is a pharmaceutical composition for inhalation comprising levosalbutamol hydrochloride and a pharmaceutically acceptable green propellant which is combination of HFO-1234ze(E) and HFA-152a. In another embodiment, disclosed is a pharmaceutical composition for inhalation comprising salbutamol sulphate and a pharmaceutically acceptable green propellant which is combination of HFO-1234ze(E) and HFA227ea. In another embodiment, disclosed is a pharmaceutical composition for inhalation comprising levosalbutamol tartrate and a pharmaceutically acceptable green propellant which is combination of HFO-1234ze(E) and HFA227ea. In another embodiment, disclosed is a pharmaceutical composition for inhalation comprising levosalbutamol hydrochloride and a pharmaceutically acceptable green propellant which is combination of HFO-1234ze(E) and HFA227ea.

In another embodiment, disclosed is a pharmaceutical composition for inhalation comprising salbutamol sulphate and a pharmaceutically acceptable green propellant which is combination of HFO-1234ze(E) and HFA-152a, wherein HFO-1234ze(E) is present in a concentration ranging from 20% to 90% by weight of composition and HFA-152a is present in a concentration ranging from 20% to 90% by weight of composition. In another embodiment, disclosed is a pharmaceutical composition for inhalation comprising salbutamol sulphate and a pharmaceutically acceptable green propellant which is combination of HFO-1234ze(E) and HFA-152a, wherein HFO-1234ze(E) is present in a concentration ranging from 70% by weight of composition and HFA-152a is present in a concentration ranging from 30% by weight of composition. In another embodiment, disclosed is a pharmaceutical composition for inhalation comprising salbutamol sulphate and a pharmaceutically acceptable green propellant which is combination of HFO-1234ze(E) and HFA-152a, wherein HFO-1234ze(E) is present in a concentration ranging from 30% by weight of composition and HFA-152a is present in a concentration ranging from 70% by weight of composition. In another embodiment, disclosed is a pharmaceutical composition for inhalation comprising levosalbutamol tartrate and a pharmaceutically acceptable green propellant which is combination of HFO-1234ze(E) and HFA-152a, wherein HFO-1234ze(E) is present in a concentration ranging from 20% to 90% by weight of composition and HFA-152a is present in a concentration ranging from 20% to 90% by weight of composition. In another embodiment, disclosed is a pharmaceutical composition for inhalation comprising levosalbutamol tartrate and a pharmaceutically acceptable green propellant which is combination of HFO-1234ze(E) and HFA-152a, wherein HFO-1234ze(E) is present in a concentration ranging from 70% by weight of composition and HFA-152a is present in a concentration ranging from 30%% by weight of composition. In another embodiment, disclosed is a pharmaceutical composition for inhalation comprising levosalbutamol tartrate and a pharmaceutically acceptable green propellant which is combination of HFO-1234ze(E) and HFA-152a, wherein HFO-1234ze(E) is present in a concentration ranging from 30%% by weight of composition and HFA-152a is present in a concentration ranging from 70% by weight of composition.

In another embodiment, disclosed is a pharmaceutical composition for inhalation comprising levosalbutamol hydrochloride and a pharmaceutically acceptable green propellant which is combination of HFO-1234ze(E) and HFA-152a, wherein HFO-1234ze(E) is present in a concentration ranging from 20% to 90% by weight of composition and HFA-152a is present in a concentration ranging from 20% to 90% by weight of composition. In another embodiment, disclosed is a pharmaceutical composition for inhalation comprising levosalbutamol hydrochloride and a pharmaceutically acceptable green propellant which is combination of HFO-1234ze(E) and HFA-152a, wherein HFO-1234ze(E) is present in a concentration ranging from 70% by weight of composition and HFA-152a is present in a concentration ranging from 30% by weight of composition. In another embodiment, disclosed is a pharmaceutical composition for inhalation comprising levosalbutamol hydrochloride and a pharmaceutically acceptable green propellant which is combination of HFO-1234ze(E) and HFA-152a, wherein HFO-1234ze(E) is present in a concentration ranging from 30% by weight of composition and HFA-152a is present in a concentration ranging from 70% by weight of composition.

In another embodiment, disclosed is a pharmaceutical composition for inhalation comprising salbutamol or enantiomer or salt or ester thereof, a pharmaceutically acceptable green propellant HFA-152a and/or HFO-1234ze(E) and a surfactant selected from group consisting of oleic acid, polyvinylpyrrolidone of different K grades, lecithin, tween 20, sorbitan trioleate, sorbitan mono oleate, tween 80, polyethylene glycol ranging from 200-1500 grade and combination thereof. In another embodiment, disclosed is a pharmaceutical composition for inhalation comprising salbutamol or enantiomer or salt or ester thereof, a pharmaceutically acceptable green propellant HFA-152a and/or HFO-1234ze(E) and polyvinylpyrrolidone having K value between 12 to 90.

In another embodiment, disclosed is a pharmaceutical composition for inhalation comprising salbutamol or enantiomer or salt or ester thereof, a pharmaceutically acceptable green propellant HFA-152a and/or HFO-1234ze(E) and polyvinylpyrrolidone having K value of 25; wherein polyvinylpyrrolidone is present in concentration between 0.001% to 0.5% by weight of composition. Alternatively, the polyvinylpyrrolidone is present in concentration of 0.001% or 0.003% or 0.005% or 0.01% or 0.03% or 0.05% or 0.07% or 0.1% or 0.2% or 0.3% or 0.4% or 0.5% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E) and polyvinylpyrrolidone having K value of 25; wherein polyvinylpyrrolidone is present in concentration between 0.005% to 0.2% by weight of composition, or between 0.01% to 0.2% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E) and polyvinylpyrrolidone having K value of 25; wherein polyvinylpyrrolidone is present in concentration of about 0.005% by weight of composition. In another embodiment, disclosed is a pharmaceutical composition for inhalation comprising levosalbutamol or salt or ester thereof, a pharmaceutically acceptable green propellant HFA-152a and/or HFO-1234ze(E) and polyvinylpyrrolidone having K value of 25; wherein polyvinylpyrrolidone is present in concentration of about 0.005% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and/or HFO-1234ze(E) and polyvinylpyrrolidone having K value of 25; wherein polyvinylpyrrolidone is present in concentration of about 0.005% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and polyvinylpyrrolidone having K value of 25; wherein polyvinylpyrrolidone is present in concentration of about 0.005% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and polyvinylpyrrolidone having K value of 25; wherein polyvinylpyrrolidone is present in concentration of about 0.005% by weight of composition; wherein HFA-152a is present in about 50% by weight of composition and HFO-1234ze(E) is present in about 50% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and polyvinylpyrrolidone having K value of 25; wherein polyvinylpyrrolidone is present in concentration of about 0.005% by weight of composition; wherein HFA-152a is present in about 30% by weight of composition and HFO-1234ze(E) is present in about 70% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and polyvinylpyrrolidone having K value of 25; wherein polyvinylpyrrolidone is present in concentration of about 0.005% by weight of composition; wherein HFA-152a is present in about 70% by weight of composition and HFO-1234ze(E) is present in about 30% by weight of composition. In another embodiment, disclosed is a pharmaceutical composition for inhalation comprising about 0.1% to about 0.5% by weight of salbutamol sulphate or levosalbutamol tartrate or levosalbutamol hydrochloride, a pharmaceutically acceptable green propellant HFA-152a and HFO-1234ze(E) and polyvinylpyrrolidone having K value of 25; wherein polyvinylpyrrolidone is present in concentration of about 0.005% by weight of composition. In another embodiment, disclosed is a pharmaceutical composition for inhalation comprising about 0.1%, or 0.2% or 0.3% or 0.4% or 0.5% by weight of salbutamol sulphate or levosalbutamol tartrate or levosalbutamol hydrochloride, a pharmaceutically acceptable green propellant HFA-152a and HFO-1234ze(E) and polyvinylpyrrolidone having K value of 25; wherein polyvinylpyrrolidone is present in concentration of about 0.005% by weight of composition.

In another embodiment, disclosed is a pharmaceutical composition for inhalation comprising salbutamol or enantiomer or salt or ester thereof, a pharmaceutically acceptable green propellant HFA-152a and/or HFO-1234ze(E) and polyethylene glycol having grade between 100 to 1000. In another embodiment, disclosed is a pharmaceutical composition for inhalation comprising salbutamol or enantiomer or salt or ester thereof, a pharmaceutically acceptable green propellant HFA-152a and/or HFO-1234ze(E) and polyethylene glycol having grade of 1000 or 400; wherein polyethylene glycol is present in concentration between 0.001% to 0.5% by weight of composition. Alternatively, the polyethylene glycol is present in concentration of 0.001% or 0.003% or 0.005% or 0.01% or 0.03% or 0.05% or 0.07% or 0.1% or 0.2% or 0.3% or 0.4% or 0.5% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E) and polyethylene glycol having grade of 1000 or 400; wherein polyethylene glycol is present in concentration between 0.005% to 0.2% by weight of composition, or between 0.01% to 0.2% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E) and polyethylene glycol having grade of 1000 or 400; wherein polyethylene glycol is present in concentration of about 0.1% by weight of composition. In another embodiment, disclosed is a pharmaceutical composition for inhalation comprising levosalbutamol or salt or ester thereof, a pharmaceutically acceptable green propellant HFA-152a and/or HFO-1234ze(E) and polyethylene glycol having grade of 1000 or 400; wherein polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and/or HFO-1234ze(E) and polyethylene glycol having grade of 1000 or 400; wherein polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and polyethylene glycol having grade of 1000 or 400; wherein polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and polyethylene glycol having grade of 1000 or 400; wherein polyethylene glycol is present in concentration of about 0.1% by weight of composition; wherein HFA-152a is present in about 50% by weight of composition and HFO-1234ze(E) is present in about 50% by weight of composition.

In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and polyethylene glycol having grade of 1000 or 400; wherein polyethylene glycol is present in concentration of about 0.1% by weight of composition; wherein HFA-152a is present in about 30% by weight of composition and HFO-1234ze(E) is present in about 70% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and polyethylene glycol having grade of 1000 or 400; wherein polyethylene glycol is present in concentration of about 0.1% by weight of composition; wherein HFA-152a is present in about 70% by weight of composition and HFO-1234ze(E) is present in about 30% by weight of composition. In another embodiment, disclosed is a pharmaceutical composition for inhalation comprising about 0.1% to about 0.5% by weight of salbutamol sulphate or levosalbutamol tartrate or levosalbutamol hydrochloride, a pharmaceutically acceptable green propellant HFA-152a and HFO-1234ze(E) and polyethylene glycol having grade of 1000 or 400; wherein polyethylene glycol is present in concentration of about 0.1% by weight of composition. In another embodiment, disclosed is a pharmaceutical composition for inhalation comprising about 0.1%, or 0.2% or 0.3% or 0.4% or 0.5% by weight of salbutamol sulphate or levosalbutamol tartrate or levosalbutamol hydrochloride, a pharmaceutically acceptable green propellant HFA-152a and HFO-1234ze(E) and polyethylene glycol having grade of 1000 or 400; wherein polyethylene glycol is present in concentration of about 0.1% by weight of composition.

In another embodiment, disclosed is a pharmaceutical composition for inhalation comprising salbutamol or enantiomer or salt or ester thereof, a pharmaceutically acceptable green propellant HFA-152a and/or HFO-1234ze(E) and sorbitan trioleate or sorbitan mono-oleate. In another embodiment, disclosed is a pharmaceutical composition for inhalation comprising salbutamol or enantiomer or salt or ester thereof, a pharmaceutically acceptable green propellant HFA-152a and/or HFO-1234ze(E) and sorbitan trioleate or sorbitan mono-oleate; wherein sorbitan trioleate or sorbitan mono-oleate is present in concentration between 0.001% to 0.5% by weight of composition. Alternatively, sorbitan trioleate or sorbitan mono-oleate is present in concentration of 0.001% or 0.003% or 0.005% or 0.01% or 0.03% or 0.05% or 0.07% or 0.1% or 0.2% or 0.3% or 0.4% or 0.5% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E) and sorbitan trioleate or sorbitan mono-oleate; wherein sorbitan trioleate or sorbitan mono-oleate is present in concentration between 0.005% to 0.2% by weight of composition, or between 0.01% to 0.2% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E) and sorbitan trioleate or sorbitan mono-oleate; wherein sorbitan trioleate or sorbitan mono-oleate is present in concentration of about 0.005% by weight of composition. In another embodiment, disclosed is a pharmaceutical composition for inhalation comprising levosalbutamol or salt or ester thereof, a pharmaceutically acceptable green propellant HFA-152a and/or HFO-1234ze(E) and sorbitan trioleate or sorbitan mono-oleate; wherein sorbitan trioleate or sorbitan mono-oleate is present in concentration of about 0.005% by weight of composition.

In another embodiment, disclosed is a pharmaceutical composition for inhalation comprising salbutamol sulphate or levosalbutamol tartrate or levosalbutamol hydrochloride, a pharmaceutically acceptable green propellant HFA-152a and/or HFO-1234ze(E) and sorbitan trioleate or sorbitan mono-oleate; wherein sorbitan trioleate or sorbitan mono-oleate is present in concentration of about 0.005% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and sorbitan trioleate or sorbitan mono-oleate; wherein sorbitan trioleate or sorbitan mono-oleate is present in concentration of about 0.005% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and sorbitan trioleate or sorbitan mono-oleate; wherein sorbitan trioleate or sorbitan mono-oleate is present in concentration of about 0.005% by weight of composition; wherein HFA-152a is present in about 50% by weight of composition and HFO-1234ze(E) is present in about 50% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and sorbitan trioleate or sorbitan mono-oleate; wherein sorbitan trioleate or sorbitan mono-oleate is present in concentration of about 0.005% by weight of composition; wherein HFA-152a is present in about 30% by weight of composition and HFO-1234ze(E) is present in about 70% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and sorbitan trioleate or sorbitan mono-oleate; wherein sorbitan trioleate or sorbitan mono-oleate is present in concentration of about 0.005% by weight of composition; wherein HFA-152a is present in about 70% by weight of composition and HFO-1234ze(E) is present in about 30% by weight of composition. In another embodiment, disclosed is a pharmaceutical composition for inhalation comprising about 0.1% to about 0.5% by weight of salbutamol sulphate or levosalbutamol tartrate or levosalbutamol hydrochloride, a pharmaceutically acceptable green propellant HFA-152a and HFO-1234ze(E) and sorbitan trioleate or sorbitan mono-oleate; wherein sorbitan trioleate or sorbitan mono-oleate is present in concentration of about 0.005% by weight of composition. In another embodiment, disclosed is a pharmaceutical composition for inhalation comprising about 0.1%, or 0.2% or 0.3% or 0.4% or 0.5% by weight of salbutamol sulphate or levosalbutamol tartrate or levosalbutamol hydrochloride, a pharmaceutically acceptable green propellant HFA-152a and HFO-1234ze(E) and sorbitan trioleate or sorbitan mono-oleate; wherein sorbitan trioleate or sorbitan mono-oleate is present in concentration of about 0.005% by weight of composition.

In another embodiment, disclosed is a pharmaceutical composition for inhalation comprising salbutamol or enantiomer or salt or ester thereof, a pharmaceutically acceptable green propellant HFA-152a and/or HFO-1234ze(E) and a surfactant and a solvent selected from group consisting of ethanol, water, propyl alcohol, isopropyl alcohol or combination thereof. In another embodiment, disclosed is a pharmaceutical composition for inhalation comprising salbutamol or enantiomer or salt or ester thereof, a pharmaceutically acceptable green propellant HFA-152a and/or HFO-1234ze(E) and polyvinylpyrrolidone having K value between 12 to 90 and ethanol. In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol and polyvinylpyrrolidone having K value of 25; wherein polyvinylpyrrolidone is present in concentration between 0.001% to 0.5% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol and polyvinylpyrrolidone having K value of 25; wherein polyvinylpyrrolidone is present in concentration between 0.005% to 0.2% by weight of composition, or between 0.01% to 0.2% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), about 1 to 15% by weight of ethanol and polyvinylpyrrolidone having K value of 25; wherein polyvinylpyrrolidone is present in concentration of about 0.005% by weight of composition. In another embodiment, disclosed is a pharmaceutical composition for inhalation comprising levosalbutamol or salt or ester thereof, a pharmaceutically acceptable green propellant HFA-152a and/or HFO-1234ze(E), about 15% by weight of ethanol and polyvinylpyrrolidone having K value of 25; wherein polyvinylpyrrolidone is present in concentration of about 0.005% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and/or HFO-1234ze(E), about 15% by weight of ethanol and polyvinylpyrrolidone having K value of 25; wherein polyvinylpyrrolidone is present in concentration of about 0.005% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol and polyvinylpyrrolidone having K value of 25; wherein polyvinylpyrrolidone is present in concentration of about 0.005% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol and polyvinylpyrrolidone having K value of 25; wherein polyvinylpyrrolidone is present in concentration of about 0.005% by weight of composition; wherein HFA-152a is present in about 50% by weight of composition and HFO-1234ze(E) is present in about 50% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol and polyvinylpyrrolidone having K value of 25; wherein polyvinylpyrrolidone is present in concentration of about 0.005% by weight of composition; wherein HFA-152a is present in about 30% by weight of composition and HFO-1234ze(E) is present in about 70% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol and polyvinylpyrrolidone having K value of 25; wherein polyvinylpyrrolidone is present in concentration of about 0.005% by weight of composition; wherein HFA-152a is present in about 70% by weight of composition and HFO-1234ze(E) is present in about 30% by weight of composition. In another embodiment, disclosed is a pharmaceutical composition for inhalation comprising about 0.1% to about 0.5% by weight of salbutamol sulphate or levosalbutamol tartrate or levosalbutamol hydrochloride, a pharmaceutically acceptable green propellant HFA-152a and HFO-1234ze(E), about 15% by weight of ethanol and polyvinylpyrrolidone having K value of 25; wherein polyvinylpyrrolidone is present in concentration of about 0.005% by weight of composition. In another embodiment, disclosed is a pharmaceutical composition for inhalation comprising about 0.1%, or 0.2% or 0.3% or 0.4% or 0.5% by weight of salbutamol sulphate or levosalbutamol tartrate or levosalbutamol hydrochloride, a pharmaceutically acceptable green propellant HFA-152a and HFO-1234ze(E), about 15% by weight of ethanol and polyvinylpyrrolidone having K value of 25; wherein polyvinylpyrrolidone is present in concentration of about 0.005% by weight of composition.

In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol and polyethylene glycol having grade between 100 to 1000. In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol and polyethylene glycol having grade of 1000 or 400; wherein polyethylene glycol is present in concentration between 0.001% to 0.5% by weight of composition. Alternatively, polyethylene glycol is present in concentration of 0.001% or 0.003% or 0.005% or 0.01% or 0.03% or 0.05% or 0.07% or 0.1% or 0.2% or 0.3% or 0.4% or 0.5% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), about 1 to 15% by weight of ethanol and polyethylene glycol having grade of 1000 or 400; wherein polyethylene glycol is present in concentration between 0.005% to 0.2% by weight of composition, or between 0.01% to 0.2% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), about 15% by weight of ethanol and polyethylene glycol having grade of 1000 or 400; wherein polyethylene glycol is present in concentration of about 0.1% by weight of composition. In another embodiment, disclosed is a pharmaceutical composition for inhalation comprising levosalbutamol or salt or ester thereof, a pharmaceutically acceptable green propellant HFA-152a and/or HFO-1234ze(E), about 15% by weight of ethanol and polyethylene glycol having grade of 1000 or 400; wherein polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and/or HFO-1234ze(E), about 15% by weight of ethanol and polyethylene glycol having grade of 1000 or 400; wherein polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E). about 15% by weight of ethanol and polyethylene glycol having grade of 1000 or 400; wherein polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol and polyethylene glycol having grade of 1000 or 400; wherein polyethylene glycol is present in concentration of about 0.1% by weight of composition; wherein HFA-152a is present in about 50% by weight of composition and HFO-1234ze(E) is present in about 50% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol and polyethylene glycol having grade of 1000 or 400; wherein polyethylene glycol is present in concentration of about 0.1% by weight of composition; wherein HFA-152a is present in about 30% by weight of composition and HFO-1234ze(E) is present in about 70% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol and polyethylene glycol having grade of 1000 or 400; wherein polyethylene glycol is present in concentration of about 0.1% by weight of composition; wherein HFA-152a is present in about 70% by weight of composition and HFO-1234ze(E) is present in about 30% by weight of composition. In another embodiment, disclosed is a pharmaceutical composition for inhalation comprising about 0.1% to about 0.5% by weight of salbutamol sulphate or levosalbutamol tartrate or levosalbutamol hydrochloride, a pharmaceutically acceptable green propellant HFA-152a and HFO-1234ze(E), about 15% by weight of ethanol and polyethylene glycol having grade of 1000 or 400; wherein polyethylene glycol is present in concentration of about 0.1% by weight of composition. In another embodiment, disclosed is a pharmaceutical composition for inhalation comprising about 0.1%, or 0.2% or 0.3% or 0.4% or 0.5% by weight of salbutamol sulphate or levosalbutamol tartrate or levosalbutamol hydrochloride, a pharmaceutically acceptable green propellant HFA-152a and HFO-1234ze(E), about 15% by weight of ethanol and polyethylene glycol having grade of 1000 or 400; wherein polyethylene glycol is present in concentration of about 0.1% by weight of composition.

In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol and sorbitan trioleate or sorbitan mono-oleate. In another embodiment, disclosed is a pharmaceutical composition for inhalation comprising salbutamol or enantiomer or salt or ester thereof, a pharmaceutically acceptable green propellant HFA-152a and/or HFO-1234ze(E), about 1 to 15% by weight of ethanol and sorbitan trioleate or sorbitan mono-oleate; wherein sorbitan trioleate or sorbitan mono-oleate is present in concentration between 0.001% to 0.5% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), about 1 to 15% by weight of ethanol and sorbitan trioleate or sorbitan mono-oleate; wherein sorbitan trioleate or sorbitan mono-oleate is present in concentration between 0.005% to 0.2% by weight of composition, or between 0.01% to 0.2% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), about 15% by weight of ethanol and sorbitan trioleate or sorbitan mono-oleate; wherein sorbitan trioleate or sorbitan mono-oleate is present in concentration of about 0.005% by weight of composition. In another embodiment, disclosed is a pharmaceutical composition for inhalation comprising levosalbutamol or salt or ester thereof, a pharmaceutically acceptable green propellant HFA-152a and/or HFO-1234ze(E), about 15% by weight of ethanol and sorbitan trioleate or sorbitan mono-oleate; wherein sorbitan trioleate or sorbitan mono-oleate is present in concentration of about 0.005% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and/or HFO-1234ze(E), about 15% by weight of ethanol and sorbitan trioleate or sorbitan mono-oleate; wherein sorbitan trioleate or sorbitan mono-oleate is present in concentration of about 0.005% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol and sorbitan trioleate or sorbitan mono-oleate; wherein sorbitan trioleate or sorbitan mono-oleate is present in concentration of about 0.005% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol and sorbitan trioleate or sorbitan mono-oleate; wherein sorbitan trioleate or sorbitan mono-oleate is present in concentration of about 0.005% by weight of composition; wherein HFA-152a is present in about 50% by weight of composition and HFO-1234ze(E) is present in about 50% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol and sorbitan trioleate or sorbitan mono-oleate; wherein sorbitan trioleate or sorbitan mono-oleate is present in concentration of about 0.005% by weight of composition; wherein HFA-152a is present in about 30% by weight of composition and HFO-1234ze(E) is present in about 70% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol and sorbitan trioleate or sorbitan mono-oleate; wherein sorbitan trioleate or sorbitan mono-oleate is present in concentration of about 0.005% by weight of composition; wherein HFA-152a is present in about 70% by weight of composition and HFO-1234ze(E) is present in about 30% by weight of composition. In another embodiment, disclosed is a pharmaceutical composition for inhalation comprising about 0.1% to about 0.5% by weight of salbutamol sulphate or levosalbutamol tartrate or levosalbutamol hydrochloride, a pharmaceutically acceptable green propellant HFA-152a and HFO-1234ze(E), about 15% by weight of ethanol and sorbitan trioleate or sorbitan mono-oleate; wherein sorbitan trioleate or sorbitan mono-oleate is present in concentration of about 0.005% by weight of composition. In another embodiment, disclosed is a pharmaceutical composition for inhalation comprising about 0.1%, or 0.2% or 0.3% or 0.4% or 0.5% by weight of salbutamol sulphate or levosalbutamol tartrate or levosalbutamol hydrochloride, a pharmaceutically acceptable green propellant HFA-152a and HFO-1234ze(E), about 15% by weight of ethanol and sorbitan trioleate or sorbitan mono-oleate; wherein sorbitan trioleate or sorbitan mono-oleate is present in concentration of about 0.005% by weight of composition.

In another embodiment, disclosed is a pharmaceutical composition for inhalation comprising salbutamol or enantiomer or salt or ester thereof, a pharmaceutically acceptable green propellant HFA-152a and/or HFO-1234ze(E), polyvinylpyrrolidone having K value between 12 to 90 and polyethylene glycol having grade between 100 to 1000. In another embodiment, disclosed is a pharmaceutical composition for inhalation comprising salbutamol or enantiomer or salt or ester thereof, a pharmaceutically acceptable green propellant HFA-152a and/or HFO-1234ze(E), polyvinylpyrrolidone having K value between 12 to 90 and polyethylene glycol having grade of 1000 or 400; wherein polyethylene glycol is present in concentration between 0.001% to 0.5% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), polyvinylpyrrolidone having K value between 12 to 90 and polyethylene glycol having grade of 1000 or 400; wherein polyethylene glycol is present in concentration between 0.005% to 0.2% by weight of composition, or between 0.01% to 0.2% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), polyvinylpyrrolidone having K value of 25 and polyethylene glycol having grade of 1000 or 400; wherein polyvinylpyrrolidone and polyethylene glycol is present in concentration of about 0.1% by weight of composition. In another embodiment, disclosed is a pharmaceutical composition for inhalation comprising levosalbutamol or salt or ester thereof, a pharmaceutically acceptable green propellant HFA-152a and/or HFO-1234ze(E), polyvinylpyrrolidone having K value of 25 and polyethylene glycol having grade of 1000 or 400; wherein polyvinylpyrrolidone and polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and/or HFO-1234ze(E), polyvinylpyrrolidone having K value of 25 and polyethylene glycol having grade of 1000 or 400; wherein polyvinylpyrrolidone and polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), polyvinylpyrrolidone having K value of 25 and polyethylene glycol having grade of 1000 or 400; wherein polyvinylpyrrolidone and polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), polyvinylpyrrolidone having K value of 25 and polyethylene glycol having grade of 1000 or 400; wherein polyvinylpyrrolidone and polyethylene glycol is present in concentration of about 0.1% by weight of composition; wherein HFA-152a is present in about 50% by weight of composition and HFO-1234ze(E) is present in about 50% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), polyvinylpyrrolidone having K value of 25 and polyethylene glycol having grade of 1000 or 400; wherein polyvinylpyrrolidone and polyethylene glycol is present in concentration of about 0.1% by weight of composition; wherein HFA-152a is present in about 30% by weight of composition and HFO-1234ze(E) is present in about 70% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), polyvinylpyrrolidone having K value of 25 and polyethylene glycol having grade of 1000 or 400; wherein polyvinylpyrrolidone and polyethylene glycol is present in concentration of about 0.1% by weight of composition; wherein HFA-152a is present in about 70% by weight of composition and HFO-1234ze(E) is present in about 30% by weight of composition. In another embodiment, disclosed is a pharmaceutical composition for inhalation comprising about 0.1% to about 0.5% by weight of salbutamol sulphate or levosalbutamol tartrate or levosalbutamol hydrochloride, a pharmaceutically acceptable green propellant HFA-152a and HFO-1234ze(E), polyvinylpyrrolidone having K value of 25 and polyethylene glycol having grade of 1000 or 400; wherein polyvinylpyrrolidone and polyethylene glycol is present in concentration of about 0.1% by weight of composition. In another embodiment, disclosed is a pharmaceutical composition for inhalation comprising about 0.1%, or 0.2% or 0.3% or 0.4% or 0.5% by weight of salbutamol sulphate or levosalbutamol tartrate or levosalbutamol hydrochloride, a pharmaceutically acceptable green propellant HFA-152a and HFO-1234ze(E), polyvinylpyrrolidone having K value of and polyethylene glycol having grade of 1000 or 400; wherein polyvinylpyrrolidone and polyethylene glycol is present in concentration of about 0.1% by weight of composition.

In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol, polyvinylpyrrolidone having K value between 12 to 90 and polyethylene glycol having grade between 100 to 1000. In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol, polyvinylpyrrolidone having K value between 12 to 90 and polyethylene glycol having grade of 1000 or 400; wherein polyethylene glycol is present in concentration between 0.001% to 0.5% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), about 1 to 15% by weight of ethanol, polyvinylpyrrolidone having K value between 12 to 90 and polyethylene glycol having grade of 1000 or 400; wherein polyethylene glycol is present in concentration between 0.005% to 0.2% by weight of composition, or between 0.01% to 0.2% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), about 15% by weight of ethanol, polyvinylpyrrolidone having K value of 25 and polyethylene glycol having grade of 1000 or 400; wherein polyvinylpyrrolidone and polyethylene glycol is present in concentration of about 0.1% by weight of composition. In another embodiment, disclosed is a pharmaceutical composition for inhalation comprising levosalbutamol or salt or ester thereof, a pharmaceutically acceptable green propellant HIFA-152a and/or HFO-1234ze(E), about 15% by weight of ethanol, polyvinylpyrrolidone having K value of 25 and polyethylene glycol having grade of 1000 or 400; wherein polyvinylpyrrolidone and polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and/or HFO-1234ze(E), about 15% by weight of ethanol, polyvinylpyrrolidone having K value of 25 and polyethylene glycol having grade of 1000 or 400; wherein polyvinylpyrrolidone and polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol, polyvinylpyrrolidone having K value of 25 and polyethylene glycol having grade of 1000 or 400; wherein polyvinylpyrrolidone and polyethylene glycol is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol, polyvinylpyrrolidone having K value of 25 and polyethylene glycol having grade of 1000 or 400; wherein polyvinylpyrrolidone and polyethylene glycol is present in concentration of about 0.1% by weight of composition; wherein HFA-152a is present in about 50% by weight of composition and HFO-1234ze(E) is present in about 50% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol, polyvinylpyrrolidone having K value of 25 and polyethylene glycol having grade of 1000 or 400; wherein polyvinylpyrrolidone and polyethylene glycol is present in concentration of about 0.1% by weight of composition; wherein HFA-152a is present in about 30% by weight of composition and HFO-1234ze(E) is present in about 70% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), about 15% by weight of ethanol, polyvinylpyrrolidone having K value of 25 and polyethylene glycol having grade of 1000 or 400; wherein polyvinylpyrrolidone and polyethylene glycol is present in concentration of about 0.1% by weight of composition; wherein HFA-152a is present in about 70% by weight of composition and HFO-1234ze(E) is present in about 30% by weight of composition. In another embodiment, disclosed is a pharmaceutical composition for inhalation comprising about 0.1% to about 0.5% by weight of salbutamol sulphate or levosalbutamol tartrate or levosalbutamol hydrochloride, a pharmaceutically acceptable green propellant HFA-152a and HFO-1234ze(E), about 15% by weight of ethanol, polyvinylpyrrolidone having K value of 25 and polyethylene glycol having grade of 1000 or 400; wherein polyvinylpyrrolidone and polyethylene glycol is present in concentration of about 0.1% by weight of composition. In another embodiment, disclosed is a pharmaceutical composition for inhalation comprising about 0.1%, or 0.2% or 0.3% or 0.4% or 0.5% by weight of salbutamol sulphate or levosalbutamol tartrate or levosalbutamol hydrochloride, a pharmaceutically acceptable green propellant HFA-152a and HFO-1234ze(E), about 15% by weight of ethanol, polyvinylpyrrolidone having K value of 25 and polyethylene glycol having grade of 1000 or 400; wherein polyvinylpyrrolidone and polyethylene glycol is present in concentration of about 0.1% by weight of composition.

In another embodiment, disclosed is a pharmaceutical composition for inhalation comprising salbutamol or enantiomer or salt or ester thereof, a pharmaceutically acceptable green propellant HFA-152a and/or HFO-1234ze(E) and oleic acid. In another embodiment, disclosed is a pharmaceutical composition for inhalation comprising salbutamol or enantiomer or salt or ester thereof, a pharmaceutically acceptable green propellant HFA-152a and/or HFO-1234ze(E) and oleic acid; wherein oleic acid is present in concentration between 0.001% to 0.5% by weight of composition. Alternatively, oleic acid is present in concentration of 0.001% or 0.003% or 0.005% or 0.01% or 0.03% or 0.05% or 0.07% or 0.1% or 0.2% or 0.3% or 0.4% or 0.5% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E) and oleic acid; wherein oleic acid is present in concentration between 0.005% to 0.2% by weight of composition, or between 0.01% to 0.2% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E) and oleic acid; wherein oleic acid is present in concentration of about 0.001 to about 0.3% by weight of composition. In another embodiment, disclosed is a pharmaceutical composition for inhalation comprising levosalbutamol or salt or ester thereof, a pharmaceutically acceptable green propellant HFA-152a and/or HFO-1234ze(E) and oleic acid; wherein oleic acid is present in concentration of about 0.03% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and/or HFO-1234ze(E) and oleic acid; wherein oleic acid is present in concentration of about 0.03% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and oleic acid; wherein oleic acid is present in concentration of about 0.03% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and oleic acid; wherein oleic acid is present in concentration of about 0.03% by weight of composition; wherein HFA-152a is present in about 50% by weight of composition and HFO-1234ze(E) is present in about 50% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and oleic acid; wherein oleic acid is present in concentration of about 0.03% by weight of composition; wherein HFA-152a is present in about 30% by weight of composition and HFO-1234ze(E) is present in about 70% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and oleic acid; wherein oleic acid is present in concentration of about 0.03% by weight of composition; wherein HFA-152a is present in about 70% by weight of composition and HFO-1234ze(E) is present in about 30% by weight of composition. In another embodiment, disclosed is a pharmaceutical composition for inhalation comprising about 0.1% to about 0.5% by weight of salbutamol sulphate or levosalbutamol tartrate or levosalbutamol hydrochloride, a pharmaceutically acceptable green propellant HFA-152a and HFO-1234ze(E) and oleic acid; wherein oleic acid is present in concentration of about 0.03% by weight of composition. In another embodiment, disclosed is a pharmaceutical composition for inhalation comprising about 0.1%, or 0.2% or 0.3% or 0.4% or 0.5% by weight of salbutamol sulphate or levosalbutamol tartrate or levosalbutamol hydrochloride, a pharmaceutically acceptable green propellant HFA-152a and HFO-1234ze(E) and oleic acid; wherein oleic acid is present in concentration of about 0.03% by weight of composition.

In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol and oleic acid. In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol and oleic acid; wherein oleic acid is present in concentration between 0.001% to 0.5% and ethanol is present in concentration between 1% to 15% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol and oleic acid; wherein oleic acid is present in concentration between 0.005% to 0.2% by weight of composition, or between 0.01% to 0.2% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E), ethanol and oleic acid; wherein oleic acid is present in concentration of about 0.001 to about 0.3% and ethanol is present in concentration between 1% to 15% by weight of composition. Alternatively, oleic acid is present in concentration of 0.001% or 0.003% or 0.005% or 0.01% or 0.03% or 0.05% or 0.07% or 0.1% or 0.2% or 0.3% or 0.4% or 0.5% by weight of composition. In another embodiment, disclosed is a pharmaceutical composition for inhalation comprising levosalbutamol or salt or ester thereof, a pharmaceutically acceptable green propellant HFA-152a and/or HFO-1234ze(E), ethanol and oleic acid; wherein oleic acid is present in concentration of about 0.03% and ethanol is present in concentration of about 15% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and/or HFO-1234ze(E), ethanol and oleic acid; wherein oleic acid is present in concentration of about 0.03% and ethanol is present in concentration of about 15% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), ethanol and oleic acid; wherein oleic acid is present in concentration of about 0.03% and ethanol is present in concentration of about 15% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), ethanol and oleic acid; wherein oleic acid is present in concentration of about 0.03% and ethanol is present in concentration of about 15% by weight of composition; wherein HFA-152a is present in about 50% by weight of composition and HFO-1234ze(E) is present in about 50% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), ethanol and oleic acid; wherein oleic acid is present in concentration of about 0.03% and ethanol is present in concentration of about 15% by weight of composition; wherein HFA-152a is present in about 30% by weight of composition and HFO-1234ze(E) is present in about 70% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E), ethanol and oleic acid; wherein oleic acid is present in concentration of about 0.03% and ethanol is present in concentration of about 15% by weight of composition; wherein HFA-152a is present in about 70% by weight of composition and HFO-1234ze(E) is present in about 30% by weight of composition. In another embodiment, disclosed is a pharmaceutical composition for inhalation comprising about 0.1% to about 0.5% by weight of salbutamol sulphate or levosalbutamol tartrate or levosalbutamol hydrochloride, a pharmaceutically acceptable green propellant HFA-152a and HFO-1234ze(E), ethanol and oleic acid; wherein oleic acid is present in concentration of about 0.03% and ethanol is present in concentration of about 15% by weight of composition. In another embodiment, disclosed is a pharmaceutical composition for inhalation comprising about 0.1%, or 0.2% or 0.3% or 0.4% or 0.5% by weight of salbutamol sulphate or levosalbutamol tartrate or levosalbutamol hydrochloride, a pharmaceutically acceptable green propellant HFA-152a and HFO-1234ze(E), ethanol and oleic acid; wherein oleic acid is present in concentration of about 0.03% and ethanol is present in concentration of about 15% by weight of composition.

In another embodiment, disclosed is a pharmaceutical composition for inhalation comprising salbutamol or enantiomer or salt or ester thereof, a pharmaceutically acceptable green propellant HFA-152a and/or HFO-1234ze(E) and magnesium stearate. In another embodiment, disclosed is a pharmaceutical composition for inhalation comprising salbutamol or enantiomer or salt or ester thereof, a pharmaceutically acceptable green propellant HFA-152a and/or HFO-1234ze(E) and magnesium stearate; wherein magnesium stearate is present in concentration between 0.001% to 0.5% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E) and magnesium stearate; wherein magnesium stearate is present in concentration between 0.01% to 0.3% by weight of composition, or between 0.01% to 0.2% by weight of composition. In another embodiment, disclosed is a pharmaceutical composition for inhalation comprising salbutamol or enantiomer or salt or ester thereof, a pharmaceutically acceptable green propellant HFA-152a and/or HFO-1234ze(E) and magnesium stearate; wherein magnesium stearate is present in concentration of about 0.001 to about 0.2% by weight of composition. In another embodiment, disclosed is a pharmaceutical composition for inhalation comprising levosalbutamol or salt or ester thereof, a pharmaceutically acceptable green propellant HFA-152a and/or HFO-1234ze(E) and magnesium stearate; wherein magnesium stearate is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and/or HFO-1234ze(E) and magnesium stearate; wherein magnesium stearate is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and magnesium stearate; wherein magnesium stearate is present in concentration of about 0.1% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and magnesium stearate; wherein magnesium stearate is present in concentration of about 0.1% by weight of composition; wherein HFA-152a is present in about 50% by weight of composition and HFO-1234ze(E) is present in about 50% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and magnesium stearate; wherein magnesium stearate is present in concentration of about 0.1% by weight of composition; wherein HFA-152a is present in about 30% by weight of composition and HFO-1234ze(E) is present in about 70% by weight of composition. In another embodiment, disclosed is a pharmaceutical composition for inhalation comprising salbutamol sulphate or levosalbutamol tartrate or levosalbutamol hydrochloride, a pharmaceutically acceptable green propellant HFA-152a and HFO-1234ze(E) and magnesium stearate; wherein magnesium stearate is present in concentration of about 0.1% by weight of composition; wherein HFA-152a is present in about 70% by weight of composition and HFO-1234ze(E) is present in about 30% by weight of composition. In another embodiment, disclosed is a pharmaceutical composition for inhalation comprising about 0.1% to about 0.5% by weight of salbutamol sulphate or levosalbutamol tartrate or levosalbutamol hydrochloride, a pharmaceutically acceptable green propellant HFA-152a and HFO-1234ze(E) and magnesium stearate; wherein magnesium stearate is present in concentration of about 0.1% by weight of composition. In another embodiment, disclosed is a pharmaceutical composition for inhalation comprising about 0.1%, or 0.2% or 0.3% or 0.4% or 0.5% by weight of salbutamol sulphate or levosalbutamol tartrate or levosalbutamol hydrochloride, a pharmaceutically acceptable green propellant HFA-152a and HFO-1234ze(E) and magnesium stearate; wherein magnesium stearate is present in concentration of about 0.1% by weight of composition.

Disclosed herein are also the process of preparing such a composition. In one embodiment, the rate of sedimentation of active agent in propellant medium depends on the process of manufacturing of the composition. In another embodiment, the compositions of this invention are manufactured by (1) sonication process, or (2) single stage filing process, or (3) dual stage filing process.

In specific embodiment of this invention, active agent (Salbutamol or enantiomer or salt or ester thereof) is mixed with some quantity of propellant (HFA-152a or HFO-1234ze(E)) or along with other excipients such as ethanol or PEG or PVP. This mixture is then subjected to sonication in order to obtain the stable suspension of active agent in propellant medium. This stable suspension is then filled in can crimped with valve and remaining part of propellant can be added to quantity sufficient. In alternate embodiment, first a homogenous slurry of active agent, some quantity of propellant and/or excipient such as ethanol, PVP, PEG, oleic acid etc mixed to obtain the homogenous slurry. This slurry may be mixed with remaining excipients and then filled into canisters crimped with valve and remaining part of propellant can be added to quantity sufficient.

In another embodiment disclosed is a sonication process comprising steps: (1) Required quantity of active agent (salbutamol or levosalbutamol) was weighed in the standard Aluminium 19 mL FCP coated canisters (C0128, Presspart, Blackbum, UK) and canister was crimped with a 63 μL valve and (2) the propellant or mixture of propellants was then filled into the cans through the valve using a manual Pamasol crimper/filler (Pamasol, Switzerland). (3) The canister is then charged with the canister with the required quantity of propellant. (4) The cans were then sonicated for 60 mins to ensure dispersion of active agent in propellant medium. (5) The propellants may be added sequentially as per the required quantity or percentage weights to obtain the final suspension composition.

In another embodiment disclosed is a sonication process comprising steps: (1) required quantity of active agent (Salbutamol or levosalbutamol) was weighed in the standard aluminium 19 mL FCP coated canisters (C0128, Presspart, Blackbum, UK) and canister was crimped with a 63 μL valve and the propellant or mixture of propellants or sequential addition of propellant was filled into the cans through the valve using a manual Pamasol crimper/filler (Pamasol, Switzerland), (2) The cans were then sonicated for 60 mins to ensure dispersion of active agent in propellant and/or excipient medium, (3) Alternatively, the propellants can be added sequentially as per the required quantity or percentage weights wherein one can dissolve excipient in one propellant and charge the propellant+excipient mixture first followed by second propellant to obtain the suspension composition, and (4) The particle size of the suspension composition is determined by conventional techniques in the art and the particle size (MMAD D50) of salbutamol or levosalbutamol in the compositions and is between 3 micron and less than 5 microns (D50).

In another embodiment disclosed is a sonication process comprising steps: (1) the suspension composition containing salbutamol Sulfate, ethanol, oleic acid and HFA-152a or HFO-1234ze(E) was prepared, (2) a stock solution of ethanol and oleic acid was prepared and salbutamol sulphate and ethanolic solution of oleic acid were weighed directly into standard aluminum 19 mL cans (C0128, Presspart, Blackbum, UK), (3) The cans were crimped with a 63 μL valve and the HFA-152a or HFO-1234ze(E) was then filled into the cans through the valve using a manual Pamasol crimper/filler (Pamasol, Switzerland), (4) The cans were then sonicated for 60 mins to ensure dispersion of active agent in ethanolic solution of oleic acid and propellant medium. The final concentration of oleic acid in the formulation was 0.03 w/w. Some samples of example 14B and 14D were weighed in 24 ml Plastic coated Glass bottle and crimped with the continuous valve. Propellant was then filled into the glass through the valve using a manual Pamasol crimper/filler (Pamasol, Switzerland), (5) The glass bottle was sonicated for 60 mins. The glass bottle was evaluated for the visual observation of suspension characteristic. This way the suspension composition of salbutamol sulfate was obtained.

In another embodiment disclosed is a single stage pressure filling for ethanol containing composition. The steps comprise:
a) Oleic acid and ethanol were dissolved in Propellant HFA-152a/HFO-1234ze(E) by homogenising this as higher speed to obtain ethanolic solution of oleic acid. This homogenised solution was transferred to the mixing vessel containing part quantity of propellant.
b) Salbutamol sulphate was homogenised with part quantity of propellant at higher speed to obtain homogenised slurry which then was transferred to the mixing vessel containing ethanolic solution of oleic acid obtained in step (a).
c) Further, mixing of composition of step (b) was done with the remaining quantity of propellant as the speed in the range of 150-300 rpm to obtain the homogeneous suspension. The homogeneous suspension was recirculated through the filling line.
d) Canister was crimped with the valves, and suspension was charged through the valve.
e) After filling the containers, they were subjected to the quarantine for valve stabilization.

In another embodiment disclosed is a dual stage pressure filling for ethanol containing composition. The steps comprise:
a) Oleic acid and ethanol were dissolved in Propellant HFA-152a/HFO-1234ze(E) by homogenising this as higher speed and this homogenised solution was transferred to the mixing vessel containing part quantity of propellant.
b) Salbutamol sulphate was homogenised with part quantity of propellant at higher speed to obtain the homogenised slurry which then was transferred to the mixing vessel containing ethanolic solution of oleic acid from step (a).
c) Further mixing of composition of step (b) was done with the part quantity of Propellant as the speed in the range of 150-300 rpm. The homogeneous suspension was recirculated through the filling line.
d) Canister was crimped with the valves, and suspension was charged through the valve.
e) Propellant was charged through the valves to make up the volume as per required fill volume.
f) After filling the containers, they were subjected to the quarantine for valve stabilization.

In yet another embodiment, disclosed is a single stage pressure filling—Dry formulation containing only propellant.
a) Salbutamol sulphate was homogenised with part quantity of propellant at higher speed to obtain the homogenised slurry which then was transferred to the mixing vessel containing part quantity of propellant.
b) Mixing was done with the remaining quantity of propellant as the speed in the range of 150-300 rpm. The homogeneous suspension was recirculated through the filling line.
c) Canister was crimped with the valves, and suspension was charged through the valve.
d) After filling the containers, they were subjected to the quarantine for valve stabilization.

In yet another embodiment, disclosed is a dual stage pressure filling—Dry formulation containing only propellant.
a) Salbutamol sulphate was homogenised with part quantity of propellant at higher speed to obtain the homogenised slurry which then was transferred to the mixing vessel containing part quantity of propellant.
b) Mixing was done with the part quantity of Propellant as the speed in the range of 150-300 rpm. The homogeneous suspension was recirculated through the filling line.
c) Canister was crimped with the valves, and suspension was charged through the valve.
d) Propellant was charged through the valves to make up the volume as per required fill volume.
e) After filling the containers, they were subjected to the quarantine for valve stabilization.

It was observed that the rate of sedimentation was comparatively better for all the formulation manufactured with Propellant HFA-152a or HFO-1234ze(E) as compared to propellant HFA-134a. Tabulated information (table 22) on the rate of sedimentation prepared by using sonication, single stage and dual stage processes is given here.

In one embodiment, the sedimentation rate of active with HFA-152a or HFO-1234ze(E) is more than 30 sec or more than 40 sec or more than 60 sec or more than 70 sec or more than 80 sec or more than 90 sec or more than 1 min, whereas the sedimentation rate of active with composition having HFA-134a or HFA227ea is less than 30 sec, or less than 20 sec.

Further disclosed here is a method of preventing or treating respiratory disorder according to any embodiment of the invention, wherein the respiratory disorder is selected from asthma, allergic asthma, hay fever, allergic rhinitis, bronchitis, emphysema, bronchiectasis, chronic obstructive pulmonary disease (COPD), adult respiratory distress syndrome (ARDS), steroid resistant asthma, severe asthma, paediatric asthma, cystic fibrosis, lung fibrosis, pulmonary fibrosis and interstitial lung disease. In further embodiments, the composition can be administered once a day or twice a dose using pMDI, or BAI or nebulizer and according to approved labelling for salbultamol or levosalbutamol.

Also disclosed here is a method of administering the invention compositions for preventing or treating respiratory disorder, wherein the respiratory disorder is selected from asthma, allergic asthma, hay fever, allergic rhinitis, bronchitis, emphysema, bronchiectasis, chronic obstructive pulmonary disease (COPD), adult respiratory distress syndrome (ARDS), steroid resistant asthma, severe asthma, paediatric asthma, cystic fibrosis, lung fibrosis, pulmonary fibrosis and interstitial lung disease. In further embodiments, the composition can be administered once a day or twice a dose using pMDI, or BAI or nebulizer and according to approved labelling for salbultamol or levosalbutamol. The invention composition is according to any embodiment of the invention.

The invention composition prepared or disclosed herein are stable compositions where the salbutamol or enantiomer or salt or ester thereof remains stable over the shelf life. Specifically, the impurities in the stable composition remain well below the standard limits as per ICH guidelines.

The invention is described in more detail by the following Examples.

### EXAMPLES

### Example 1

TABLE 1

| | % w/w | | | | | | | |
|---|---|---|---|---|---|---|---|---|
| Ingredient | A | B | C | D | E | F | G | H |
| Salbutamol Sulphate | 0.1% | 0.2% | 0.3% | 0.4% | — | — | — | — |
| Levosalbutamol tartrate | — | — | — | — | 0.1% | 0.2% | 0.3% | 0.4% |

| | |
|---|---|
| Propellant HFA-152a | q.s. to 100% |

Manufacturing process: Required quantity of active agent (salbutamol or levosalbutamol) was weighed in the standard Aluminum 19 mL FCP coated canisters (C0128, Presspart, Blackburn, UK) and canister was crimped with a 63 μL valve and the propellant or mixture of propellants was then filled into the cans through the valve using a manual Pamasol crimper/filler (Pamasol, Switzerland). The canister is then charged with the canister with the required quantity of propellant. The cans were then sonicated for 60 mins to ensure dispersion of active agent in propellant medium. The propellants may be added sequentially as per the required quantity or percentage weights to obtain the final suspension composition. This process was used for preparing composition of Example 1 to 9. The particle size of the suspension composition is determined by conventional techniques in the art and the particle size (MMAD D50) of salbutamol or levosalbutamol in the compositions of 1 to 9 ranges between 3 micron and less than 5 microns (D50).

### Example 2

TABLE 2

| | % w/w | | | | | | | |
|---|---|---|---|---|---|---|---|---|
| Ingredient | A | B | C | D | E | F | G | H |
| Salbutamol Sulphate | 0.1% | 0.2% | 0.3% | 0.4% | — | — | — | — |
| Levosalbutamol tartrate | — | — | — | — | 0.1% | 0.2% | 0.3% | 0.4% |

| | |
|---|---|
| Propellant HFA-152a | q.s. to 50% |
| Propellant HFA134a | q.s. to 50% |

### Example 3

TABLE 3

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| Propellant | q.s. to 100% | | | | | | | |
| HFO- | | | | | | | | |
| 1234ze(E) | | | | | | | | |

### Example 4

TABLE 4

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| Propellant | q.s. to 50% | | | | | | | |
| HFO- | | | | | | | | |
| 1234ze(E) | | | | | | | | |
| Propellant | q.s. to 50% | | | | | | | |
| HFA-152a | | | | | | | | |

### Example 5

TABLE 5

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| Propellant | q.s. to 30% | | | | | | | |
| HFO- | | | | | | | | |
| 1234ze(E) | | | | | | | | |
| Propellant | q.s. to 70% | | | | | | | |
| HFA-152a | | | | | | | | |

### Example 6

TABLE 6

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| Propellant | q.s. to 70% | | | | | | | |
| HFO- | | | | | | | | |
| 1234ze(E) | | | | | | | | |
| Propellant | q.s. to 30% | | | | | | | |
| HFA-152a | | | | | | | | |

### Example 7

TABLE 7

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |

| | |
|---|---|
| Propellant | q.s. to 50% |
| HFO- | |
| 1234ze(E) | |
| Propellant | q.s. to 50% |
| HFA134a | |

### Example 8

TABLE 8

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| Propellant | q.s. to 70% | | | | | | | |
| HFO- | | | | | | | | |
| 1234ze(E) | | | | | | | | |
| Propellant | q.s. to 30% | | | | | | | |
| HFA134a | | | | | | | | |

### Example 9

TABLE 9

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| Propellant | q.s. to 30% | | | | | | | |
| HFO- | | | | | | | | |
| 1234ze(E) | | | | | | | | |
| Propellant | q.s. to 70% | | | | | | | |
| HFA134a | | | | | | | | |

### Example 10

TABLE 10

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| PVP | 0.005% | | | | | | | |
| Propellant | q.s. to 100% | | | | | | | |
| HFO- | | | | | | | | |
| 1234ze(E) | | | | | | | | |
| or Propellant | | | | | | | | |
| HFA-152a | | | | | | | | |

### Manufacturing Process:

HFA-134a is a very poor solvent which fails to dissolve the commonly used surfactants sorbitan trioleate, sorbitan mono-oleate, lecithin and oleic acid in useful concentrations without the aid of a co-solvent. Thus, solubility studies were conducted with various surfactants/stabilizers (oleic acid, PVP, lecithin, tween 20, sorbitan trioleate, sorbitan mono oleate, tween 80, PEG ranging from 200-1500 grade/viscosity modifiers (glycerol)/organic (ascorbic acid, citric acid, malic acid) and inorganic acids (HCl, sulfuric acid, nitric acid) and evaluated for visual observation. The evaluated excipients were found to be soluble in HFA-152a and HFO-1234ze(E) as compared to the propellant HFA-134a. The solubility was evaluated with and without co-solvent such as ethanol. The surfactants were found to be soluble with alcohol in the concentration ranging from 1% to 15%. First required quantities of excipients (PVP, ethanol, PEG, oleic acid, Sorbitan Trioleate, Sorbitan mono-oleate, magnesium stearate) were weighed and dissolved in propellant or mixture of propellants (50/50% or 70/30% or 30/70%). Required quantity of active agent (Salbutamol or levosalbutamol) was weighed in the standard aluminum 19 mL FCP coated canisters (C0128, Presspart, Blackburn, UK) and canister was crimped with a 63 μL valve and the propellant or mixture of propellants or sequential addition of propellant was filled into the cans through the valve using a manual Pamasol crimper/filler (Pamasol, Switzerland). The cans were then sonicated for 60 mins to ensure dispersion of active agent in propellant and/or excipient medium. Alternatively, the propellants can be added sequentially as per the required quantity or percentage weights wherein one can dissolve excipient in one propellant and charge the propellant+excipient mixture first followed by second propellant to obtain the suspension composition. This process was used for preparing suspension composition of Example 9 to Example 12 and Example 15 to 19. The particle size of the suspension composition is determined by conventional techniques in the art and the particle size (MMAD D50) of salbutamol or levosalbutamol in the compositions of 1 to 8 ranges between 3 micron and less than 5 microns (D50).

### Example 11

TABLE 11

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| PVP | 0.005% | | | | | | | |
| Ethanol | 15% | | | | | | | |
| Propellant | q.s. to 100% | | | | | | | |
| HFO- | | | | | | | | |
| 1234ze(E) | | | | | | | | |
| or Propellant | | | | | | | | |
| HFA-152a | | | | | | | | |

### Example 12

TABLE 12

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| PEG 1000 or | 0.1% | | | | | | | |
| PEG400 | | | | | | | | |
| Propellant | q.s. to 100% | | | | | | | |
| HFO- | | | | | | | | |
| 1234ze(E) | | | | | | | | |
| or Propellant | | | | | | | | |
| HFA-152a | | | | | | | | |

### Example 13

TABLE 13

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| PEG 1000 or | 0.1% | | | | | | | |
| PEG400 | | | | | | | | |
| Ethanol | 15% | | | | | | | |
| Propellant | q.s. to 100% | | | | | | | |
| HFO- | | | | | | | | |
| 1234ze(E) | | | | | | | | |
| or Propellant | | | | | | | | |
| HFA-152a | | | | | | | | |

### Example 14

TABLE 14

| | % w/w | | | |
|---|---|---|---|---|
| | A | | | |
| Ingredient | (Comparative Example) | B | C | D |
| Salbutamol Sulphate | 100 mcg/spray (0.1667%) | 100 mcg/spray (0.21%) | 100 mcg/spray (0.16%) | 100 mcg/spray (0.16%) |
| Oleic Acid | — | 0.03% | — | 0.03% |
| Ethanol | — | 15% | — | 15% |
| Propellant HFA-152Aa | — | q.s. to100% (Volume: 16 ml) | — | — |
| Propellant HFO- | — | — | q.s. to 100% (Volume: 16 ml) | q.s. to 100% (Volume: 16 ml) |
| 1234ze(E) | | | | |
| Propellant HFA134a | q.s. to 100% (Volume: 15 ml) | — | — | — |

### Manufacturing Process:

Example 14B and 14D: The suspension composition containing salbutamol Sulfate, ethanol, oleic acid and HFA-152a or HFO-1234ze(E) was prepared. A stock solution of ethanol and oleic acid was prepared. Salbutamol sulphate and ethanolic solution of oleic acid were weighed directly into standard aluminum 19 mL cans (C0128, Presspart, Blackbum, UK). The cans were crimped with a 63 μL valve and the HFA-152a or HFO-1234ze(E) was then filled into the cans through the valve using a manual Pamasol crimper/filler (Pamasol, Switzerland). The cans were then sonicated for 60 mins to ensure dispersion of active agent in ethanolic solution of oleic acid and propellant medium. The final concentration of oleic acid in the formulation was 0.03 w/w. Some samples of example 14B and 14D were weighed in 24 ml Plastic coated Glass bottle and crimped with the continuous valve. Propellant was then filled into the glass through the valve using a manual Pamasol crimper/filler (Pamasol, Switzerland). The glass bottle was sonicated for 60 mins. The glass bottle was evaluated for the visual observation of suspension characteristic. This way the suspension composition of salbutamol sulfate was obtained.

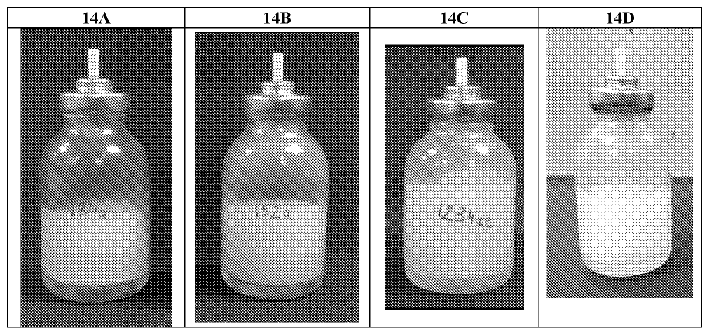

Example 14A and 14C: The comparative example composition in 14A is prepared as per process of example 1. Some samples of both 14A and 14C were charged in plastic coated glass bottle crimped with the continuous valve for visual characterization. This way the suspension composition of salbutamol Sulfate was obtained. The images of glass bottles of composition 13A, B, C and D are shown in FIG. 1.

### Example 15

| | % w/w | | | |
|---|---|---|---|---|
| | A | | | |
| Ingredient | (Comparative Example) | B | C | D |
| Levosalbutamol Sulphate | 100 mcg/spray (0.1667%) | 100 mcg/spray (0.21%) | 100 mcg/spray (0.16%) | 100 mcg/spray (0.16%) |
| Oleic Acid | — | 0.03% | — | 0.03% |
| Ethanol | — | 15% | — | 15% |
| Propellant HFA-152a | — | q.s. to100% (Volume: 16 ml) | — | — |
| Propellant HFO- | — | — | q.s. to 100% (Volume: 16 ml) | q.s. to 100% (Volume: 16 ml) |
| 1234ze(E) | | | | |
| Propellant HFA 134a | q.s. to 100% (Volume: 15 ml) | — | — | — |

Example 15 is prepared as per the process described in example 14.

### Example 16

TABLE 16

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| Sorbitan | 0.005% | | | | | | | |
| Trioleate/ | | | | | | | | |
| Sorbitan mono- | | | | | | | | |
| oleate | | | | | | | | |
| Propellant | q.s. to 100% | | | | | | | |
| HFO- | | | | | | | | |
| 1234ze(E) | | | | | | | | |
| or Propellant | | | | | | | | |
| HFA-152a | | | | | | | | |

### Example 17

TABLE 17

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| Sorbitan | 0.005% | | | | | | | |
| Trioleate/ | | | | | | | | |
| Sorbitan mono- | | | | | | | | |
| oleate | | | | | | | | |
| Ethanol | 15% | | | | | | | |
| Propellant | q.s. to 100% | | | | | | | |
| HFO- | | | | | | | | |
| 1234ze(E) | | | | | | | | |
| or Propellant | | | | | | | | |
| HFA-152a | | | | | | | | |

### Example 18

TABLE 18

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| PEG 1000 or | 0.1% | | | | | | | |
| PEG 400 | | | | | | | | |
| PVP | 0.005% | | | | | | | |
| Propellant | q.s. to 100% | | | | | | | |
| HFO- | | | | | | | | |
| 1234ze(E) | | | | | | | | |
| or Propellant | | | | | | | | |
| HFA-152a | | | | | | | | |

### Example 19

TABLE 19

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| PVP-K25 | 0.005% | | | | | | | |
| PEG 1000 or | 0.1% | | | | | | | |
| PEG 400 | | | | | | | | |
| Ethanol | 15% | | | | | | | |
| Propellant | q.s. to 100% | | | | | | | |
| HFO- | | | | | | | | |
| 1234ze(E) | | | | | | | | |
| or Propellant | | | | | | | | |
| HFA-152a | | | | | | | | |

### Example 20

TABLE 20

| | % w/w | | | | | | | |
|---|---|---|---|---|---|---|---|---|
| Ingredient | A | B | C | D | E | F | G | H |
| Salbutamol | 0.1% | 0.2% | 0.3% | 0.4% | — | — | — | — |
| Sulphate | | | | | | | | |
| Levosal- | — | — | — | — | 0.1% | 0.2% | 0.3% | 0.4% |
| butamol | | | | | | | | |
| tartrate | | | | | | | | |
| Mg. stearate | 0.1% | | | | | | | |
| Propellant | q.s. to 100% | | | | | | | |
| HFO- | | | | | | | | |
| 1234ze(E) | | | | | | | | |
| or Propellant | | | | | | | | |
| HFA-152a | | | | | | | | |

### Example 21

The pharmaceutical compositions prepared as per example 14 were subjected to measurement of visual and physicochemical characterization such as delivered dose uniformity and fine particle mass using methods known in the art such as Anderson cascade impactor (ACI, Copley Scientific, Nottingham UK) which was connected to a vacuum pump (Copley Scientific, Nottingham UK). For each experiment, four actuations of the can were discharged into the ACI at 28.3 L·min-1 as per pharmacopeia guidelines. Following aerosolization, the ACI apparatus was dismantled and the actuator and each part of the ACI was washed down into known volumes of the HPLC mobile phase. The mass of drug deposited on each part of the ACI was determined by HPLC. This protocol was repeated on three cans, following which, the fine particle mass (FPM) and emitted dose were determined, and results summarized in below table.

TABLE 21
Physicochemical performance of composition of Example 13

| | 14A | | | |
|---|---|---|---|---|
| | (Comparative | | | |
| | Example) | 14B | 14C | 14D |
| Emitted dose in mcg | 86.0 | 86.6 | 87.3 | 86.4 |
| % RSD | 9.0 | 3.3 | 5.4 | 4.1 |
| FPM in mcg (Particle below 5 μm) | 38.5 | 42.3 | 39.1 | 39.7 |
| % RSD | 7.6 | 3.5 | 6.2 | 6.2 |

*RSD: Relative Standard Deviation.

It is clear the visual observation, from FIG. 1, indicates rate of sedimentation is lower for the suspension formulated with both HFA-152a and HFO-1234ze(E) as compared to the propellant HFA134a. Further as per table 21, indicates the rate of sedimentation is lower for the suspension formulated with propellants HFA-152a and HFO 1234ze as compared to the propellant HFA134a. This helped in minimising the variability which is evident from the % RSD observed during delivered dose uniformity and aerodynamic particle size distribution (APSD) testing. The change in propellant has no impact on the targeted delivered dose uniformity and APSD.

### Example 22: Alternate Process of Preparing the Propellant Compositions

To evaluate the performance of the rate of sedimentation, the suspension of salbutamol sulphate was manufactured with the varying manufacturing processes such as single stage and dual stage filling.

### Single Stage Pressure Filling—Ethanol Formulation

a) Oleic acid and ethanol were dissolved in Propellant HFA-152a/HFO-1234ze(E) by homogenising this as higher speed to obtain ethanolic solution of oleic acid. This homogenised solution was transferred to the mixing vessel containing part quantity of propellant.
b) Salbutamol sulphate was homogenised with part quantity of propellant at higher speed to obtain homogenised slurry which then was transferred to the mixing vessel containing ethanolic solution of oleic acid obtained in step (a).
c) Further, mixing of composition of step (b) was done with the remaining quantity of propellant as the speed in the range of 150-300 rpm to obtain the homogeneous suspension. The homogeneous suspension was recirculated through the filling line.
d) Canister was crimped with the valves, and suspension was charged through the valve.
e) After filling the containers, they were subjected to the quarantine for valve stabilization.

### Dual Stage Pressure Filling—Ethanol Formulation

a) Oleic acid and ethanol were dissolved in Propellant HFA-152a/HFO-1234ze(E) by homogenising this as higher speed and this homogenised solution was transferred to the mixing vessel containing part quantity of propellant.
b) Salbutamol sulphate was homogenised with part quantity of propellant at higher speed to obtain the homogenised slurry which then was transferred to the mixing vessel containing ethanolic solution of oleic acid from step (a).
c) Further mixing of composition of step (b) was done with the part quantity of Propellant as the speed in the range of 150-300 rpm. The homogeneous suspension was recirculated through the filling line.
d) Canister was crimped with the valves, and suspension was charged through the valve.
e) Propellant was charged through the valves to make up the volume as per required fill volume.
f) After filling the containers, they were subjected to the quarantine for valve stabilization.

Single stage pressure filling—Dry formulation containing only propellant.
a) Salbutamol sulphate was homogenised with part quantity of propellant at higher speed to obtain the homogenised slurry which then was transferred to the mixing vessel containing ethanolic solution of oleic acid.
b) Mixing was done with the remaining quantity of propellant as the speed in the range of 150-300 rpm. The homogeneous suspension was recirculated through the filling line.
c) Canister was crimped with the valves, and suspension was charged through the valve.
d) After filling the containers, they were subjected to the quarantine for valve stabilization.

Dual stage pressure filling—Ethanol formulation
a) Salbutamol sulphate was homogenised with part quantity of propellant at higher speed to obtain the homogenised slurry which then was transferred to the mixing vessel containing ethanolic solution of oleic acid.
b) Mixing was done with the part quantity of Propellant as the speed in the range of 150-300 rpm. The homogeneous suspension was recirculated through the filling line.
c) Canister was crimped with the valves, and suspension was charged through the valve.
d) Propellant was charged through the valves to make up the volume as per required fill volume.
e) After filling the containers, they were subjected to the quarantine for valve stabilization.

The rate of sedimentation was comparatively better for all the formulation manufactured with Propellant HFA-152a or HFO-1234ze(E) as compared to propellant HFA-134a. Below given is the tabulated information (table 22) on the rate of sedimentation prepared by using sonication, single stage and dual stage processes.

TABLE 22

| | | Manufacturing | Rate of | |
|---|---|---|---|---|
| No | Formulation | process | sedimentation | |
| A | Salbutamol Sulfate/Oleic | Sonication | 32 | sec |
| | acid/Ethanol/HFA-152a | Process | | |
| B | Salbutamol Sulfate/Oleic | Single stage | 57 | sec |
| | acid/Ethanol/HFA-152a | fpressure illing | | |
| C | Salbutamol Sulfate/Oleic | Dual stage | 65 | sec |
| | acid/Ethanol/HFA-152a | fpressure illing | | |
| D | Salbutamol Sulfate/Oleic | Sonication | 33 | sec |
| | acid/Ethanol/HFO-1234ze(E) | Process | | |
| E | Salbutamol Sulfate/Oleic | Single stage | 67 | sec |
| | acid/Ethanol/HFO-1234ze(E) | pressure filling | | |
| F | Salbutamol Sulfate/Oleic | Dual stage | 78 | sec |
| | acid/Ethanol/HFO-1234ze(E) | pressure filling | | |
| G | Salbutamol Sulfate/HFO- | Sonication | 33 | sec |
| | 1234ze(E) | Process | | |
| H | Salbutamol Sulfate/HFO- | Single stage | 63 | sec |
| | 1234ze(E) | pressure filling | | |
| I | Salbutamol Sulfate/HFO- | Dual stage | 72 | sec |
| | 1234ze(E) | pressure filling | | |
| J | Salbutamol Sulfate/HFA- | Sonication | 7 | Sec |
| | 134a | Process | | |
| K | Salbutamol Sulfate/HFA- | Single stage | 10 | sec |
| | 134a | fpressure illing | | |
| L | Salbutamol Sulfate/HFA- | Dual stage | 12 | sec |

| | |
|---|---|
| 134a | pressure filling |

1. A pharmaceutical composition for inhalation comprising salbutamol or an enantiomer or salt or ester thereof, a pharmaceutically acceptable green propellant HFA-152a and/or HFO-1234ze(E), ethanol and oleic acid.

2. The pharmaceutical composition of claim 1; wherein the oleic acid is present in a concentration between about 0.001% to about 0.5% by weight of composition.

3. The pharmaceutical composition of claim 2; wherein the oleic acid is present in the concentration of about 0.03% by weight of composition.

4. The pharmaceutical composition of claim 1; wherein the ethanol is present in the concentration between 1% to 15% by weight of composition.

5. The pharmaceutical composition of claim 4; wherein the ethanol is present in the concentration of about 15% by weight of composition.

6. The pharmaceutical composition of claim 1, wherein the composition is administered using a pressurized metered dose inhaler or a breath actuated inhaler or a nebulizer.

7. The pharmaceutical composition of claim 1, wherein the composition is in the form of a suspension or a solution.

8. The pharmaceutical composition of claim 1, wherein salbutamol or enantiomer or salt or ester thereof is salbutamol base, salbutamol sulphate, levosalbutamol base, or levosalbutamol tartrate or levosalbutamol hydrochloride.

9. The pharmaceutical composition of claim 8, wherein the salbutamol base or salbutamol sulphate is present in the concentration ranging from 0.05% w/w to 1% w/w or preferably between 0.1% w/w to 0.5% w/w or more preferably between 0.2% w/w to 0.4% w/w of the composition.

10. The pharmaceutical composition of claim 8, wherein the levosalbutamol or levalbuterol sulphate or levalbuterol tartrate or levalbuterol hydrochloride is present in the concentration ranging from 0.05% w/w to 1% w/w or preferably between 0.1% w/w to 0.5% w/w or more preferably between 0.2% w/w to 0.4% w/w of the composition.

11. The pharmaceutical composition of claim 1, wherein the pharmaceutically acceptable green propellant is HFA-152a.

12. The pharmaceutical composition of claim 11, wherein HFA-152a is present in a concentration of at least 30% or 40% or 50% or 60% or 70% or 80% or 90% or 99% in the composition.

13. The pharmaceutical composition of claim 12, wherein HFA-152a is present in a concentration of at least 90% in the composition.

14. The pharmaceutical composition of claim 11, wherein the composition contains less than 1000 ppm of water based on the total weight of the pharmaceutical composition.

15. The pharmaceutical composition of claim 14, wherein the composition contains less than 0.5 ppm of water based on the total weight of the pharmaceutical composition.

16. The pharmaceutical composition of claim 11, wherein the composition contains less than 400 ppm of oxygen based on the total weight of the pharmaceutical composition.

17. The pharmaceutical composition of claim 16, wherein the composition contains less than 1 ppm of oxygen based on the total weight of the pharmaceutical composition.

18. The pharmaceutical composition of claim 11, wherein the composition contains less than 0.5 ppm of oxygen based on the total weight of the pharmaceutical composition.

19. The pharmaceutical composition of claim 11, wherein the propellant HFA-152a is more than 90% or 95% or 99% or 99.5% or 99.99% pure and does not have any substantial impurities or is free from any impurities.

20. The pharmaceutical composition of claim 11, wherein the HFA-152a is sole propellant in the composition.

21. The pharmaceutical composition of claim 1, wherein the pharmaceutically acceptable green propellant HFO-1234ze(E).

22. The pharmaceutical composition of claim 21, wherein the HFO-1234ze(E) is present in the concentration of at least 30% or 40% or 50% or 60% or 70% or 80% or 90% or 99% in the composition.

23. The pharmaceutical composition of claim 22, wherein the HFO-1234ze(E) is present in the concentration of at least 90% in the composition.

24. The pharmaceutical composition of claim 21, wherein the composition contains less than 400 ppm of water based on the total weight of the pharmaceutical composition.

25. The pharmaceutical composition of claim 24, wherein the composition contains less than 0.5 ppm of water based on the total weight of the pharmaceutical composition.

26. The pharmaceutical composition of claim 21, wherein the composition contains less than 1000 ppm of oxygen based on the total weight of the pharmaceutical composition.

27. The pharmaceutical composition of claim 26, wherein the composition contains less than 1 ppm of oxygen based on the total weight of the pharmaceutical composition.

28. The pharmaceutical composition of claim 21, wherein the composition contains less than 0.5 ppm of oxygen based on the total weight of the pharmaceutical composition.

29. The pharmaceutical composition of claim 21, wherein the propellant HFO-1234ze(E) is more than 90% or 95% or 99% or 99.5% or 99.99% pure and does not have any substantial impurities or is free from any impurities.

30. The pharmaceutical composition of claim 29, wherein the propellant HFO-1234ze(E) contains less than 0.5 to 10 ppm of unsaturated impurities or less than 0.25 ppm of unsaturated impurities.

31. The pharmaceutical composition of claim 29, wherein the propellant HFO-1234ze(E) contains less than 10 ppm of HFO-1234ze(Z).

32. The pharmaceutical composition of claim 31, wherein the propellant HFO-1234ze(E) contains less than 5 ppm of HFO-1234ze(Z).

33. The pharmaceutical composition of claim 11, wherein the composition when administered using the metered dose inhaler produces a fine particle mass of the salbutamol or enantiomer or salt or ester thereof with less variability having relative standard deviation of less than 6% over shelf life as compared to the composition comprising salbutamol or enantiomer or salt or ester thereof and a propellant HFA-134a or HFA-227ea.

34. The pharmaceutical composition of claim 21, wherein the composition when administered using the metered dose inhaler produces a fine particle mass of the salbutamol or enantiomer or salt or ester thereof with less variability having relative standard deviation of less than 6.5% over shelf life as compared to the composition comprising salbutamol or enantiomer or salt or ester thereof and a propellant HFA-134a or HFA-227ea.

35. A pharmaceutical composition for inhalation comprising salbutamol sulphate or levosalbutamol tartrate or levosalbutamol hydrochloride, a pharmaceutically acceptable green propellant HFA-152a and HFO-1234ze(E) and oleic acid; wherein oleic acid is present in concentration of about 0.03% by weight of composition.

36. The pharmaceutical composition of claim 35, wherein (a) HFA-152a is present in about 50% by weight of composition and HFO-1234ze(E) is present in about 50% by weight of composition or (b) HFA-152a is present in about 30% by weight of composition and HFO-1234ze(E) is present in about 70% by weight of composition, or (c) HFA-152a is present in about 70% by weight of composition and HFO-1234ze(E) is present in about 30% by weight of composition.

37. The pharmaceutical composition of claim 35, wherein the composition further comprises ethanol and is present in the concentration between 1% to 15% by weight of composition.

38. The pharmaceutical composition of claim 37; wherein the ethanol is present in the concentration of about 15% by weight of composition.

39. A pharmaceutical composition for inhalation comprising salbutamol sulphate or levosalbutamol tartrate or levosalbutamol hydrochloride, a pharmaceutically acceptable green propellant HFA-152a and HFO-1234ze(E), ethanol and oleic acid; wherein oleic acid is present in concentration of about 0.03% and ethanol is present in concentration of about 15% by weight of composition.

40. The pharmaceutical composition of claim 39, wherein (a) HFA-152a is present in about 50% by weight of composition and HFO-1234ze(E) is present in about 50% by weight of composition, or (b) HFA-152a is present in about 30% by weight of composition and HFO-1234ze(E) is present in about 70% by weight of composition, or (c) HFA-152a is present in about 70% by weight of composition and HFO-1234ze(E) is present in about 30% by weight of composition.
